(19) United States
(12) Patent Application Publication
(10) Pub. No.: US 2025/0195899 A1
(43) Pub. Date: Jun. 19, 2025

(54) PACING OUTPUT OPTIMIZATION TO IMPROVE DEVICE LONGEVITY

(71) Applicant: Pacesetter, Inc., Sylmar, CA (US)

(72) Inventors: Alexander R. Bornzin, Ventura, CA (US); Matthew G. Fishler, Scotts Valley, CA (US); Gabriel A. Mouchawar, Valencia, CA (US); Luke C. McSpadden, Studio City, CA (US)

(73) Assignee: Pacesetter, Inc., Sylmar, CA (US)

(21) Appl. No.: 18/981,237

(22) Filed: Dec. 13, 2024

Related U.S. Application Data
(60) Provisional application No. 63/610,833, filed on Dec. 15, 2023.

(51) Int. Cl. A61N 1/37; A61N 1/372; A61N 1/375; G16H 50/70

(52) U.S. Cl. CPC A61N 1/3712; A61N 1/37247; A61N 1/3756; G16H 50/70

(57) ABSTRACT

Disclosed herein are methods for use with an IMD configured to deliver pacing pulses to cardiac tissue, and related systems for use with and/or including an IMD. A method includes determining a pacing impedance of the cardiac tissue, a first capture threshold of the cardiac tissue, and an estimate of a maximum membrane response for the cardiac tissue. Additionally, the method includes using the maximum membrane response to determine an iso-safety factor strength duration curve. The method also includes determining a current or charge drain curve, and determining, based on the iso-safety factor strength duration curve and the current or charge drain curve, a preferred pacing parameter set that includes a preferred pulse width and a preferred pacing amplitude, which provides a specified safety margin.

### PRIORITY CLAIM

The present application claims priority to U.S. Provisional Patent Application No. 63/610,833, filed Dec. 15, 2023, which is incorporated herein by reference in its entirety.

### FIELD OF TECHNOLOGY

Embodiments described herein generally relate leadless pacemakers (LPs) and other types of implantable medical devices (IMDs) that are capable of delivering pacing, as well as external devices that can be used to program or otherwise adjust pacing parameters of such IMDs. Embodiments described herein also relate to methods for use with IMDs and external devices, and to systems including IMDs and/or external devices.

### BACKGROUND

LPs and other types IMDs that are capable of delivering pacing each include a battery having a fixed amount of charge available for their operations including providing pacing therapy. Accordingly, reducing battery current drain is important for prolonging device longevity. This is especially important in the case of LPs which have smaller batteries than conventional pacemakers, thereby enabling the necessary formfactor for transvenous delivery and implantation within cardiac chambers.

### SUMMARY

Certain embodiments of the present technology are directed to methods for use with an implantable medical device (IMD) configured to deliver pacing pulses to cardiac tissue of a patient's heart. In an embodiment, the method comprises determining a pacing impedance of the cardiac tissue that is to be paced by the IMD and determining a first capture threshold of the cardiac tissue that is to be paced by the IMD when the cardiac tissue is paced using one or more pacing pulses having a first pulse width. The method further comprises determining an estimate of a maximum membrane response for the cardiac tissue of the patient's heart, based on the pacing impedance, the first pulse width, and the first capture threshold with or without a specified safety margin applied, wherein the first pulse width and the capture threshold with the specified safety margin applied, collectively comprise a first pacing parameter set. Additionally, the method comprises determining an iso-safety factor strength duration curve by determining, based on the first pacing parameter set and the estimate of the maximum membrane response, a plurality of further pacing parameter sets that each includes a respective different pulse width and a respective pacing amplitude having the specified safety margin applied. The method also comprises determining a current or charge drain curve by determining, based on the pacing impedance, a respective current or charge drain value for each of the first pacing parameter set and the plurality of further pacing parameter sets. The method further comprises determining, based on the iso-safety factor strength duration curve and the current or charge drain curve, a preferred pacing parameter set that includes a preferred pulse width and a preferred pacing amplitude, which provides the specified safety margin at a lower current or charge drain than the first pacing parameter set.

In certain embodiments, determining the iso-safety factor strength duration curve includes: determining a strength duration curve by determining, based on the first pacing parameter set and the estimate of the maximum membrane response, a plurality of further pacing parameter sets that each includes the respective different pulse width and a respective pacing amplitude without having the specified safety margin applied; and applying the safety margin to respective pacing amplitudes of the first pacing parameter set and the plurality of further pacing parameter sets to thereby determine further respective pacing amplitudes with the safety margin applied, which are included in the iso-safety factor strength duration curve.

In other embodiments, determining the iso-safety factor strength duration curve includes determining, based on the first pacing parameter set and the estimate of the maximum membrane response, the plurality of further pacing parameter sets that each includes the respective different pulse width and a respective pacing amplitude having the specified safety margin applied.

In certain embodiments, determining the estimate of the maximum membrane response for the cardiac tissue of the patient's heart is also based on a membrane time constant.

In certain embodiments, determining the iso-safety factor strength duration curve is performed without determining any additional capture threshold besides the first capture threshold. In certain such embodiments, the membrane time constant is determined based on the first capture threshold and empirical patient population data.

In certain embodiments, the method further comprises determining one or more additional capture thresholds that correspond respectively to one or more additional pulse widths, and determining the membrane time constant based on the first capture threshold and the one or more additional capture thresholds. In certain such embodiments, determining the membrane time constant based on the first capture threshold and the one or more additional capture thresholds includes performing error minimization using a nonlinear least squares regression algorithm.

In certain embodiments, the method further comprises determining one or more additional capture thresholds that correspond respectively to one or more additional pulse widths, thereby resulting in multiple capture thresholds with the specified safety margin applied being determined, wherein the determining the iso-safety factor strength duration curve is based on the multiple capture thresholds with the specified safety margin applied.

In certain embodiments, the method further comprises: determining the membrane time constant based on the multiple capture thresholds with the specified safety margin applied, and using the membrane time constant to produce the iso-safety factor strength duration curve. In certain such embodiments, determining the membrane time constant based on the multiple capture thresholds with the specified safety margin applied includes performing error minimization using a nonlinear least squares regression algorithm.

In certain embodiments, the method further comprises the IMD delivering pacing pulses, having the preferred pulse width and the preferred pacing amplitude, to the cardiac tissue of the patient's heart.

In certain embodiments, the method further comprises displaying, via a user interface of an external device that is configured to communicate with the IMD, information indicative of the preferred pacing parameter set that includes the preferred pulse width and the preferred pacing amplitude is displayed to a user, and programming the IMD to use the preferred pacing parameter set.

In certain embodiments, determining the preferred pacing parameter set that includes the preferred pulse width and the preferred pacing amplitude is performed by an external device that is configured to communicate with the IMD.

In certain embodiments, determining the preferred pacing parameter set that includes the preferred pulse width and the preferred pacing amplitude is performed by the IMD.

In certain embodiments, the IMD includes a battery that outputs a battery voltage and a charge pump that can apply one of a plurality of multiplier factors to the battery voltage to achieve a range of pacing amplitudes, and determining the current or charge drain curve is performed in a manner that accounts for the plurality of multiplier factors and causes the current or charge drain curve to have multiple inflection points.

In certain embodiments, the IMD comprises a leadless pacemaker (LP) that includes at least two electrodes that are used to deliver the pacing pulses to the cardiac tissue of a patient's heart. In certain such embodiments, the method is performed for each of a plurality of LPs to thereby determine a different said preferred pacing parameter set for each of the plurality of LPs.

In certain embodiments, the IMD comprises a pacemaker to which one or more leads having one or more electrodes is/are attached, and the method is performed for each of a plurality of different electrode combinations to thereby determine a different said preferred pacing parameter set for each of the plurality of different electrode combinations.

Certain embodiments of the present technology are directed to a system including or for use with an IMD configured to deliver pacing pulses to cardiac tissue of a patient's heart. The system comprises one or more processors configured to: determine a pacing impedance of the cardiac tissue that is to be paced by the IMD; determine a first capture threshold of the cardiac tissue that is to be paced by the IMD when the cardiac tissue is paced using one or more pacing pulses having a first pulse width; determine an estimate of a maximum membrane response for the cardiac tissue of the patient's heart, based on the pacing impedance, the first pulse width, and the first capture threshold with or without a specified safety margin applied; wherein the first pulse width and the capture threshold with the specified safety margin applied, collectively comprise a first pacing parameter set; determine an iso-safety factor strength duration curve by determining, based on the first pacing parameter set and the estimate of the maximum membrane response, a plurality of further pacing parameter sets that each includes a respective different pulse width and a respective pacing amplitude having the specified safety margin applied; determine a current or charge drain curve by determining, based on the pacing impedance, a respective current or charge drain value for each of the first pacing parameter set and the plurality of further pacing parameter sets; and determine, based on the iso-safety factor strength duration curve and the current or charge drain curve, a preferred pacing parameter set that includes a preferred pulse width and a preferred pacing amplitude, which provides the specified safety margin at a lower current or charge drain than the first pacing parameter set.

In certain embodiments, the one or more processors is/are configured to: determine a strength duration curve by determining, based on the first pacing parameter set and the estimate of the maximum membrane response, a plurality of further pacing parameter sets that each includes the respective different pulse width and a respective pacing amplitude without having the specified safety margin applied; and apply the safety margin to respective pacing amplitudes of the first pacing parameter set and the plurality of further pacing parameter sets to thereby determine further respective pacing amplitudes with the safety margin applied, which are included in the iso-safety factor strength duration curve.

In certain embodiments, the one or more processors is/are configured to determine, based on the first pacing parameter set and the estimate of the maximum membrane response, the plurality of further pacing parameter sets that each includes the respective different pulse width and a respective pacing amplitude having the specified safety margin applied, to thereby determine the iso-safety factor strength duration curve.

In certain embodiments, the one or more processors is/are configured to determine the estimate of the maximum membrane response for the cardiac tissue of the patient's heart is also based on a membrane time constant.

In certain embodiments, the system is configured to program the IMD to deliver pacing pulses, having the preferred pulse width and the preferred pacing amplitude, to the cardiac tissue of the patient's heart.

In certain embodiments, the system further comprises: plurality of implantable electrodes; and an implantable pulse generator electrically couplable to the implantable electrodes; wherein at least one of the one or more processors are configured to control the pulse generator to deliver pacing pulses to cardiac tissue via at least two of the implantable electrodes. In certain such embodiments, the system comprises the IMD that includes or is coupled to the plurality of implantable electrodes. In certain such embodiments, the one or more processors is/are included in the IMD.

In certain embodiments, the system comprises an external device and at least one of the one or more processors configured to determine the preferred pacing parameter set is/are included in the external device. In certain such embodiments, the external device is configured to communicate with the IMD and to program the IMD to use the preferred pacing parameter set to pace the cardiac tissue. In certain such embodiments, information indicative of the preferred pacing parameter set that includes the preferred pulse width and the preferred pacing amplitude is displayed to a user via a user interface of the external device that is configured to communicate with the IMD, and the external device is configured to program the IMD to use the preferred pacing parameter set in response to the user accepting the preferred pacing parameter set via the user interface.

In certain embodiments, the IMD includes a battery that outputs a battery voltage and a charge pump that can apply one of a plurality of multiplier factors to the battery voltage to achieve a range of pacing amplitudes, and the one or more processors are configured to determine the current or charge drain curve in a manner that accounts for the plurality of multiplier factors and causes the current or charge drain curve to have multiple inflection points. In certain embodiments, the IMD comprises a leadless pacemaker.

This summary is not intended to be a complete description of the embodiments of the present technology. Other features and advantages of the embodiments of the present technology will appear from the following description in which the preferred embodiments have been set forth in detail, in conjunction with the accompanying drawings and claims.

### BRIEF DESCRIPTION OF THE DRAWINGS

Embodiments of the present technology relating to both structure and method of operation may best be understood by referring to the following description and accompanying drawings, in which similar reference characters denote similar elements throughout the several views:

FIG. 1 illustrates a system formed in accordance with certain embodiments herein as implanted in a heart.

FIG. 2 is a block diagram of a single LP in accordance with certain embodiments herein.

FIG. 3 illustrates an LP in accordance with certain embodiments herein.

FIG. 4A is a graph that illustrates an example strength-duration curve and an example iso-safety factor strength duration curve, wherein the graph has pacing pulse width (aka stimulus duration) plotted on the horizontal axis and pacing pulse amplitude (aka stimulus intensity) plotted on the vertical axis.

FIG. 4B is a graph that illustrates an example pacing current drain curve, wherein the graph has pacing pulse width plotted on the horizontal axis and pacing current drain plotted on the vertical axis.

FIG. 4C is a graph that includes the example strength duration curve and the example iso-safety factor strength duration curve introduced in FIG. 4A, as well as the example pacing current drain curve introduced in FIG. 4B, and illustrates how such curves can be used to identify a preferred pacing parameter set in accordance with certain embodiments of the present technology.

FIG. 5A is a graph that illustrates example membrane response curves.

FIG. 5B is a graph that illustrates normalized versions of the membrane response curves introduced in FIG. 5A

FIGS. 6A and 6B are high level flow diagrams that is used to summarize methods according to certain embodiments of the present technology.

FIG. 7 is an example user interface of an external device that can be used to perform certain steps or functions of certain embodiments of the present technology.

FIG. 8 shows a block diagram of one embodiment of an IMD (e.g., LP, conventional pacemaker, or ICD) that is implanted into a patient as part of the implantable cardiac system in accordance with certain embodiments herein.

FIG. 9 shows a block diagram of one embodiment of an external device for use in communicating with and/or programming an IMD, and which can be used to implement certain embodiments of the present technology.

### DETAILED DESCRIPTION

Capture of cardiac tissue during pacing therapy is achieved through electrical stimulation pulses (aka pacing pulses) at a combination of pulse amplitude and pulse width, wherein the pulse amplitude and the pulse width of pacing pulses can be referred to collectively a pacing parameter set (aka a set of pacing parameters). In practice, pacing parameters are selected above a capture threshold needed for cardiac stimulation by a corresponding safety margin appropriate for the patient. Using an optimal combination of pulse amplitude and pulse width that stimulates capture while minimizing current drain from the pacemaker battery can increase longevity of an IMD by reducing and preferably minimizing an amount of charge depleted from a battery of the IMD, such as an LP, during delivery of pacing pulses.

Certain embodiments of the present technology take advantage of various curves and models (e.g., provided by one or more algorithms) to provide a recommended a pacing parameter set for an LP or other type of IMD capable of delivering pacing, wherein the recommended pacing parameter set provides a capture safety margin and reduces pacing charge consumption, thereby extending device longevity.

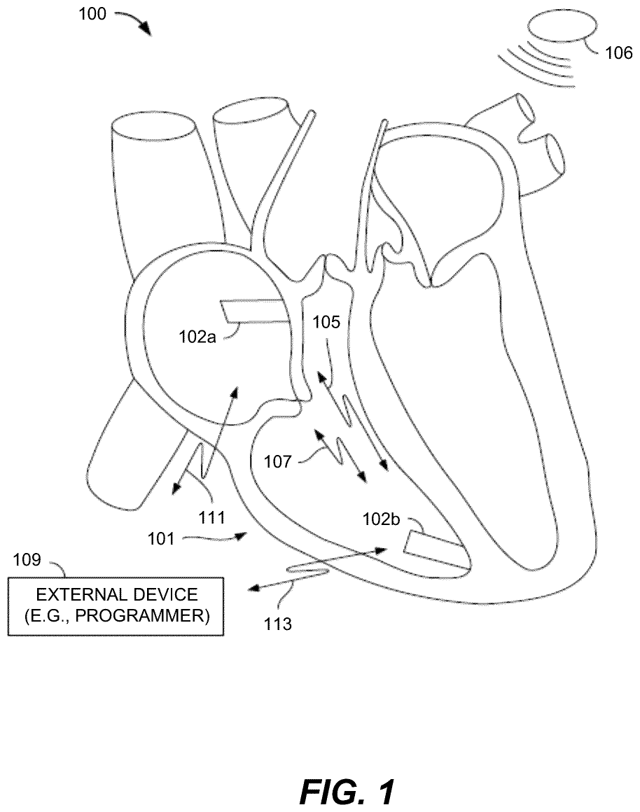

However, before providing addition details of specific embodiments of the present technology, an exemplary system in or with which embodiments of the present technology can be used will first be described with reference to FIGS. 1-3. More specifically, FIGS. 1-3 will be used to describe an example dual chamber LP system, which optionally also includes a non-vascular implantable cardioverter defibrillator (NV-ICD), such as a subcutaneous-ICD (S-ICD), and an external device (e.g., a programmer).

FIG. 1 illustrates a system 100 that is configured to be implanted in a heart 101. The system 100 includes LPs 102a and 102b located in different chambers of the heart. LP 102a is located in a right atrium, and thus can also be referred to herein as an atrial LP (aLP). The LP 102b is located in a right ventricle, and thus can also be referred to herein as a ventricular LP (vLP). The aLP 102a and the vLP 102b can be referred to collectively herein as the LPs 102, or individually as an LP 102. The LPs 102a and 102b can communicate with one another to inform one another of various local physiologic activities, such as local intrinsic events, local paced events and the like. The LPs 102a and 102b may be constructed in a similar manner, but operate differently based upon which chamber the LP 102a or 102b is located.

In certain embodiments, LPs 102a and 102b communicate with one another, and/or with an ICD 106, by conductive communication through the same electrodes that are used for sensing and/or delivery of pacing therapy. The LPs 102a and 102b may also be able to use conductive communication to communicate with an external device, e.g., a programmer 109, having electrodes placed on the skin of a patient within with the LPs 102a and 102b are implanted. The LPs 102a and 102b can each alternatively, or additionally, include an antenna that would enable them to communicate with one another, the ICD 106 and/or an external device, using RF communication. Alternatively, or additionally, it is possible that the LPs 102a, 102b utilize another type of communication, such as inductive communication, in which case the LPs 102a, 102b can each include a respective inductive communication coil. While only two LPs are shown in FIG. 1, it is possible that more than two LPs can be implanted in a patient. For example, to provide for bi-ventricular pacing and/or cardiac resynchronization therapy (CRT), in addition to having LPs implanted in the right atrial (RA) chamber and the right ventricular (RV) chamber, a further LP can be implanted in the left ventricular (LV) chamber.

Each LP 102 uses two or more electrodes located within, on, or within a few centimeters of the housing of the LP, for pacing and sensing at the cardiac chamber. Where the LPs 102 communication using conductive communication, the electrodes of the LPs 102 can also be used for bidirectional conductive communication with one another, as well as with the programmer 109, and the ICD 106.

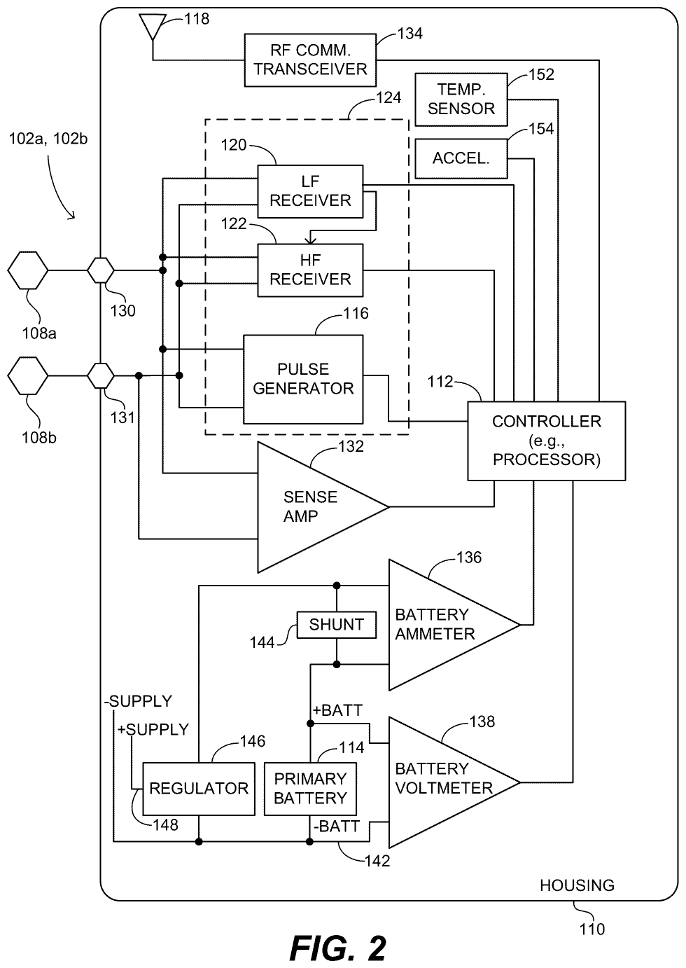

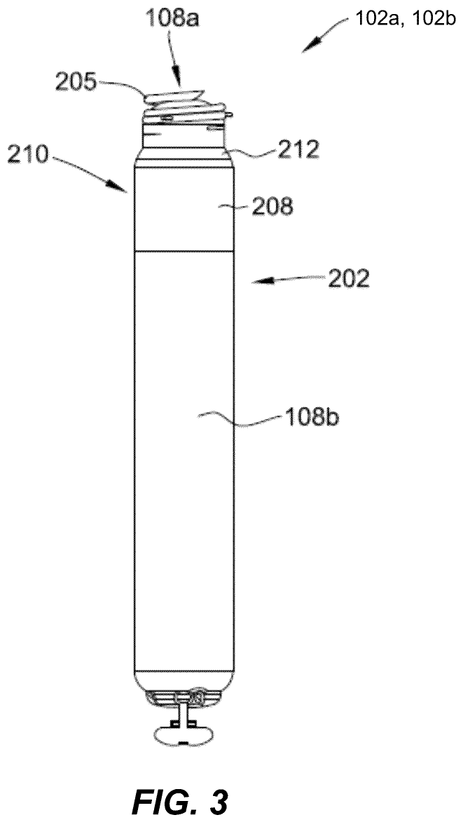

Referring to FIG. 2, a block diagram shows an embodiment for portions of the electronics within LPs 102a, 102b configured to provide conductive communication through the sensing/pacing electrode. One or more of LPs 102a and 102b include at least two leadless electrodes configured for delivering cardiac pacing pulses, sensing evoked and/or natural cardiac electrical signals, and uni-directional or bi-directional conductive communication. In FIG. 2 (and FIG. 3) the two electrodes shown therein are labeled 108a and 108b. Such electrodes can be referred to collectively as the electrodes 108, or individually as an electrode 108. An LP 102, or other type of IMD, can include more than two electrodes 108, depending upon implementation.

In FIG. 2, each of the LPs 102a, 102b is shown as including first and second receivers 120 and 122 that collectively define separate first and second conductive communication channels 105 and 107 (FIG. 1), (among other things) between LPs 102a and 102b. Although first and second receivers 120 and 122 are depicted, in other embodiments, each LP 102a, 102b may only include the first receiver 120, or more generally may include only a single receiver that is configured to receive conductive communication signals. It is also possible that an LP 102 may include additional receivers other than first and second receivers 120 and 122. The pulse generator 116 can function as a transmitter that transmits i2i communication signals using the electrodes 108.

In accordance with certain embodiments, when one of the LPs 102a and 102b senses an intrinsic event or delivers a paced event, the corresponding LP 102a, 102b transmits an implant event message to the other LP 102a, 102b. For example, when an aLP 102a senses/paces an atrial event, the aLP 102a transmits an implant event message including an event marker indicative of a nature of the event (e.g., intrinsic/sensed atrial event, paced atrial event). When a vLP 102b senses/paces a ventricular event, the vLP 102b transmits an implant event message including an event marker indicative of a nature of the event (e.g., intrinsic/sensed ventricular event, paced ventricular event). In certain embodiments, each LP 102a, 102b transmits an implant event message to the other LP 102a, 102b preceding the actual pace pulse so that the remote LP can blank its sense inputs in anticipation of that remote pace pulse (to prevent inappropriate crosstalk sensing). The above describe implant event messages are examples of implant-to-implant (i2i) messages.

Still referring to FIG. 2, the LP 102 is also shown as including an antenna 118 that is coupled to a radio frequency (RF) communication transceiver 134, which is configured to transmit and receive RF communication messages using an RF communication protocol, such as a Bluetooth protocol, WiFi protocol, Bluetooth low energy (BLE) protocol, Medical Device Radiocommunications Service (MedRadio) protocol, and/or the like. In certain embodiments, the antenna 118 can be integrated into a fixation mechanism (e.g., 205) of the LP, in which case the antenna can be referred to as a fixation antenna. The RF communication transceiver 134 consumes more battery power than the conductive communication transceiver 124. More generally, it is more power efficient from an LP 102 (or other type of IMD) to use conductive communication than to use RF communication to communicate with another LP 102 (or other type of IMD). Accordingly, in accordance with certain embodiments of the present technology, the LP 102 (or other type of IMD) is configured to primarily transmit and receive messages using conductive communication, and RF communication is used as a backup or auxiliary type of communication that can be used when conductive communication is deactivated (aka turned off) or is unsuccessful or otherwise deficient, as will be described in additional detail below.

It is possible that the LPs 102a, 102b are only capable of utilizing conductive communication for communicating with one another and/or other devices (such as a programmer 109 and/or ICD 106), in which case the antenna 118 and the RF communication transceiver 134 may be eliminated. It may also be the case that the LPs 102a, 102b are only configured to communicate using RF communication, and to not utilize conductive communication, in which case certain circuitry may be eliminated, such as the receivers 120, 122. Alternatively, or additionally, it is possible that the LPs 102a, 102b utilize another type of communication, such as inductive communication, in which case the LPs 102a, 102b can each include a respective inductive communication coil.

In accordance with certain embodiments herein, the external device 109 may communicate with LP 102a, 102b utilizing one or more of the above described communication schemes, i.e., using conductive communication, RF communication, and/or inductive communication.

In some embodiments, each individual LP 102 can comprise a hermetic housing 110 configured for placement on or attachment to the inside or outside of a cardiac chamber and at least two leadless electrodes 108 proximal to the housing 110 and configured for bidirectional communication with at least one other device (e.g., an NV-ICD 106) within or outside the body.

FIG. 2 depicts a single LP 102 (e.g., the LP 102a or 102b) and shows the LP's functional elements substantially enclosed in a hermetic housing 110. The LP 102 has at least two electrodes 108 located within, on, or near the housing 110, for delivering pacing pulses to and sensing electrical activity from the muscle of the cardiac chamber, and for bidirectional communication with at least one other device within or outside the body. Hermetic feedthroughs 130, 131 conduct electrode signals through the housing 110. The housing 110 contains a primary battery 114 to supply power for pacing, sensing, and communication. The housing 110 also contains circuits 132 for sensing cardiac activity from the electrodes 108, receivers 120, 122 for receiving information from at least one other device via the electrodes 108, and the pulse generator 116 for generating pacing pulses for delivery via the electrodes 108 and also for transmitting information to at least one other device via the electrodes 108. The housing 110 can further contain circuits for monitoring device health, for example a battery current monitor 136 and a battery voltage monitor 138, and can contain circuits for controlling operations in a predetermined manner. While one pulse generator 116 is shown in FIG. 2, it is possible that that LP includes multiple pulse generators, one of which is used for producing pacing pulses, and another one of which is used for producing conductive communication pulses. The pulse generator that produces pacing pulses can include a charge pump to provide for a wide range of pacing pulse amplitudes, as will be described in additional details below.

The electrodes 108 can be configured to communicate bidirectionally among the multiple LPs and/or the implanted ICD 106 to coordinate pacing pulse delivery and optionally other therapeutic or diagnostic features using messages that identify an event at an individual pacemaker originating the message and a pacemaker receiving the message react as directed by the message depending on the origin of the message. An LP 102a, 102b that receives the event message reacts as directed by the event message depending on the message origin or location. In some embodiments or conditions, the two or more leadless electrodes 108 can be configured to communicate bidirectionally among the one or more LPs 102 and/or the ICD 106 and transmit data including designated codes for events detected or created by an individual pacemaker. Individual pacemakers can be configured to issue a unique code corresponding to an event type and a location of the sending pacemaker.

Referring again to FIGS. 1 and 2, the cardiac pacing system 100 may comprise an ICD 106 in addition to one or more LPs 102a, 102b configured for implantation in electrical contact with a cardiac chamber and for performing cardiac rhythm management functions in combination with the implantable ICD 106. The implantable ICD 106 and the one or more LPs 102a, 102b configured for leadless intercommunication by information conduction through body tissue and/or wireless transmission between transmitters and receivers in accordance with the embodiments discussed herein.

In a further embodiment, a cardiac pacing system 100 comprises at least one LP 102a, 102b configured for implantation in electrical contact with a cardiac chamber and configured to perform cardiac pacing functions in combination with the co-implanted ICD 106. Each LP 102 comprise at least two leadless electrodes 108 configured for delivering cardiac pacing pulses, sensing evoked and/or natural cardiac electrical signals, and transmitting information to the co-implanted ICD 106.

As shown in the illustrative embodiments, an LP 102a, 102b can comprise two or more leadless electrodes 108 configured for delivering cardiac pacing pulses, sensing evoked and/or natural cardiac electrical signals, and bidirectionally communicating with the co-implanted ICD 106.

Each LP 102a, 102b can be configured for operation in a respective particular location and to have a respective particular functionality at manufacture and/or by programming by an external programmer. Bidirectional communication among the multiple LPs can be arranged to communicate notification of a sensed heartbeat or delivered pacing pulse event and encoding type and location of the event to another implanted pacemaker or pacemakers. The LP 102a, 102b receiving the communication decode the information and respond depending on location of the receiving pacemaker and predetermined system functionality.

In some embodiments, the LPs 102a and 102b are configured to be implantable in any chamber of the heart, namely either atrium (RA, LA) or either ventricle (RV, LV). Furthermore, for dual-chamber configurations, multiple LPs may be co-implanted (e.g., one in the RA and one in the RV, one in the RV and one in the coronary sinus proximate the LV). Certain pacemaker parameters and functions depend on (or assume) knowledge of the chamber in which the LP is implanted (and thus with which the LP is interacting; e.g., pacing and/or sensing). Some non-limiting examples include an evoked response algorithm, use of AF suppression in a local chamber, blanking & refractory periods, etc. Accordingly, each LP should know an identity of the chamber in (or on) which the LP is implanted, and processes may be implemented to automatically identify a local chamber associated with each LP.

Also shown in FIG. 2, the primary battery 114 has positive terminal 140 and negative terminal 142. Current from the positive terminal 140 of primary battery 114 flows through a shunt 144 to a regulator circuit 146 to create a positive voltage supply 148 suitable for powering the remaining circuitry of the LP 102. The shunt 144 enables the battery current monitor 136 to provide the controller 112 with an indication of battery current drain and indirectly of device health. The illustrative power supply can be a primary battery 114. The battery 114 produces a battery voltage (V_{battery}) that can be measured, e.g., using the battery voltmeter 138. A charge pump of the pulse generator 116 can be used to produce pacing pulse of various different amplitudes based on the battery voltage, as described in additional detail below.

Still referring to FIG. 2, the LP is shown as including a temperature sensor 152. The temperature sensor can be any one of various different types of well-known temperature sensors, or can be a future developed temperature sensor. For one example, the temperature sensor 152 can be a thermistor, a thermocouple, a resistance thermometer, or a silicon bandgap temperature sensor, but is not limited thereto. Regardless of how the temperature sensor 152 is implemented, it is preferably that the temperature sensed by the sensor is provided to the controller 112 as a digital signal indicative of the blood temperature of the patient within which the LP is implanted. The temperature sensor 152 can be hermetically sealed within the housing 110, but that need not be the case. The temperature sensor 152 can be used in various manners. For example, the temperature sensor 152 can be used to detect an activity level of the patient to adjust a pacing rate, i.e., for use in rate responsive pacing.

Referring to FIG. 2, the LP is also shown as including an accelerometer 154 which can be hermetically contained within the housing 110. The accelerometer 154 can be any one of various different types of well-known accelerometers, or can be a future developed accelerometer. For one example, the accelerometer 154 can be or include, e.g., a MEMS (micro-electromechanical system) multi-axis accelerometer of the type exploiting capacitive or optical cantilever beam techniques, or a piezoelectric accelerometer that employs the piezoelectric effect of certain materials to measure dynamic changes in mechanical variables. For example, the accelerometer 154 can be used to detect an activity level and/or posture of the patient to adjust a pacing rate, e.g., for use in rate responsive pacing. It would also be possible to use outputs of both the accelerometer 154 and the temperature sensor 152 to monitor the activity level of a patient.

In various embodiments, LP 102a, 102b can manage power consumption to draw limited power from the battery, thereby reducing device volume. Each circuit in the system can be designed to avoid large peak currents. For example, cardiac pacing can be achieved by discharging a tank capacitor (not shown) across the pacing electrodes. Recharging of the tank capacitor is typically controlled by a charge pump circuit. In a particular embodiment, the charge pump circuit is throttled to recharge the tank capacitor at constant power from the battery. The charge pump circuit, which can be referred to more generally as the charge pump, is a type of DC-DC converter that leverages switched-capacitor techniques to either increase or decrease a voltage level that is output by the battery of the LP. More generally, the charge pump can be used to adjust the voltage level (Vout) that is output by the battery by one or more multiples, e.g., such as 0.5, 1.0, 1.5, 2.0, or 3.0. That is why charge pumps are sometimes referred to as multipliers. Such a charge pump can be part of the pulse generator 116, as noted above.

FIG. 3 shows an example form factor of the LPs 102a, 102b. Each LP can include a hermetic housing 202 (e.g., 110 in FIG. 2) with electrodes 108a and 108b disposed thereon. As shown, electrode 108a can be separated from but surrounded partially by a fixation mechanism 205, and the electrode 108b can be disposed on the housing 202. The fixation mechanism 205 can be a fixation helix, a plurality of hooks, barbs, or other attaching features configured to attach the LP to tissue, such as heart tissue. As noted above, an antenna (e.g., 118) can be at least partially implanted by or as part of the fixation mechanism. The electrodes 108a and 108b are examples of the electrodes 108 shown in and discussed above with reference to FIG. 2.

The housing can also include an electronics compartment 210 within the housing that contains the electronic components necessary for operation of the LP, including, e.g., a pulse generator, transceiver, a battery, and a processor for operation. The hermetic housing 202 can be adapted to be implanted on or in a human heart, and can be cylindrically shaped, rectangular, spherical, or any other appropriate shapes, for example.

The housing can comprise a conductive, biocompatible, inert, and anodically safe material such as titanium, 316L stainless steel, or other similar materials. The housing can further comprise an insulator disposed on the conductive material to separate electrodes 108a and 108b. The insulator can be an insulative coating on a portion of the housing between the electrodes, and can comprise materials such as silicone, polyurethane, parylene, or another biocompatible electrical insulator commonly used for implantable medical devices. In the embodiment of FIG. 2, a single insulator 208 is disposed along the portion of the housing between electrodes 108a and 108b. In some embodiments, the housing itself can comprise an insulator instead of a conductor, such as an alumina ceramic or other similar materials, and the electrodes can be disposed upon the housing.

As shown in FIG. 3, the LP can further include a header assembly 212 to isolate 108a and 108b. The header assembly 212 can be made from PEEK, tecothane or another biocompatible plastic, and can contain a ceramic to metal feedthrough, a glass to metal feedthrough, or other appropriate feedthrough insulator as known in the art.

The electrodes 108a and 108b can comprise pace/sense electrodes, or return electrodes. A low-polarization coating can be applied to the electrodes, such as sintered platinum, platinum-iridium, iridium, iridium-oxide, titanium-nitride, carbon, or other materials commonly used to reduce polarization effects, for example. In FIG. 2, electrode 108a can be a pace/sense electrode and electrode 108b can be a return electrode. The electrode 108b can be a portion of the conductive housing 202 that does not include an insulator 208.

Several techniques and structures can be used for attaching the housing 202 to the interior or exterior wall of the heart. A helical fixation mechanism 205, can enable insertion of the device endocardially or epicardially through a guiding catheter. A torqueable catheter can be used to rotate the housing and force the fixation device into heart tissue, thus affixing the fixation device (and also the electrode 108a in FIG. 2) into contact with stimulable tissue. Electrode 108b can serve as an indifferent electrode for sensing and pacing. The fixation mechanism may be coated partially or in full for electrical insulation, and a steroid-eluting matrix may be included on or near the device to minimize fibrotic reaction, as is known in conventional pacing electrode-leads.

### Pacing Output Optimization

Increasing and preferably maximizing the longevity of IMDs capable of delivering pacing, such as LPs and conventional pacemakers, is important, especially for LPs having a small battery. In accordance with certain embodiments, by determining an iso-safety factor strength duration curve for a patient, which applies a safety margin (aka a safety factor), and also determining a current (or charge) drain curve, a preferred pacing parameter set (including a preferred pulse width, and a preferred pulse amplitude) that reduces and preferably minimizes current drain from the battery of the IMD resulting from pacing can be determined. The preferred pacing parameter set can also be referred to herein as a recommended pacing parameter set.

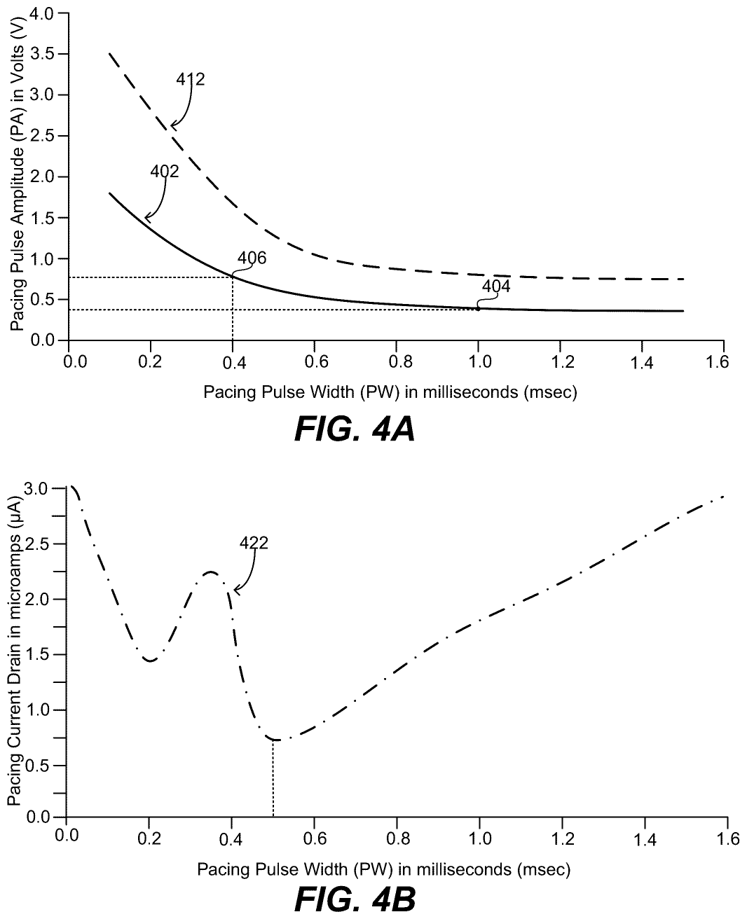

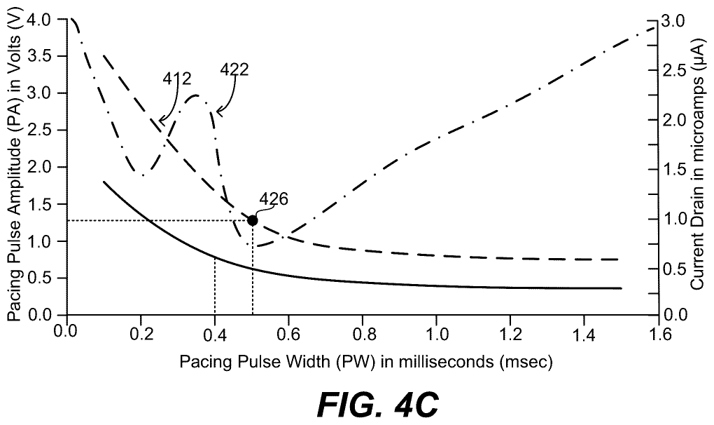

For a pacemaker to induce a response in excitable myocardial tissue, the pacing stimulus must be of sufficient amplitude and duration to initiate a cascade of self-propagating depolarizing wavefronts to “capture” local myocardial tissue. This minimal stimulus intensity, used to cause capture, is known as a capture threshold. The relationship between minimal pacing stimulus intensity (aka pulse amplitude) and duration (aka pulse width) has been described using a strength-duration curve. Where a safety margin (aka safety factor) is applied to such a strength-duration curve, the resulting curve is referred to as an iso-safety factor strength duration curve. FIG. 4A is a graph that illustrates an example strength-duration curve 402, and an example iso-safety factor strength duration curve 412, wherein the graph has pacing pulse width (aka stimulus duration) plotted on the horizontal axis and pacing pulse amplitude (aka stimulus intensity) plotted on the vertical axis. The example strength-duration curve 402 is a representation of the relationship between pulse amplitude and pulse width to achieve capture for an IMD capable of delivering pacing pulses to a patient. The phrase strength-duration curve, as used herein, is also intended to cover the information indicative of the strength duration curve, i.e., pacing pulse amplitudes and corresponding pulse widths that define points along the curve strength duration curve. Similarly, the phrase iso-safety factor strength-duration curve, as used herein, is also intended to cover the information indicative of the iso-safety factor strength duration curve, i.e., pacing pulse amplitudes and corresponding pulse widths that define points along the iso-safety factor strength duration curve.

Still referring to FIG. 4A, based on the strength-duration curve 402, the rheobase 404 can be determined, wherein the rheobase is the lowest pulse amplitude at which capture will occur, with no further reduction in the capture threshold being obtained by a further increase in pulse width. Explained another, the rheobase is the pulse amplitude corresponding to the point on the strength-duration curve beyond which the capture threshold no longer decreases with an increasing pulse width. Thus, in FIG. 4A, the rheobase 404 is at about 0.4 V. Additionally, based on the strength-duration curve 402, the chronaxie (aka the chronaxie time) can be determined, wherein the chronaxie is the pulse width that corresponds to the pulse amplitude at twice the rheobase. Thus, in FIG. 4A, the chronaxie 406, which is the pulse width at 0.8 V (because 0.4 V×2=0.8 V) is about 0.4 msec.

Still referring to FIG. 4A, the iso-safety factor strength duration curve 412 is produced by applying a safety margin (aka a safety factor) to the strength-duration curve 402. The safety margin can be a multiplier that is applied by multiplying the pacing voltage (corresponding to the capture threshold) by the multiplier, or the safety margin can be a value that is additive applied by adding a voltage to the pacing voltage (corresponding to the capture threshold). For some examples, applying a multiplier safety marker of 1.4 to the pacing voltage (corresponding to the capture threshold) effectively doubles (i.e., increases by 100%) the stimulation energy (since 1.4\hat{ }2≈2); applying a multiplier safety margin of 1.7 to the pacing voltage (corresponding to the capture threshold) effectively triples the stimulation energy (since 1.7\hat{ }2≈3); and applying a multiplier safety marking of 2 (i.e., doubling the pacing voltage corresponding to the capture threshold) effectively quadruples the stimulation energy (since 2\hat{ }2=4). Where the safety margin is a multiplier, it can be within a specified range from 1.2 to 2.0, but is not limited thereto. Where the safety margin is additive, it can be within a specified range, e.g., from 1.0 V to 3.0 V, but is not limited thereto. In FIG. 4A, the iso-safety factor strength duration curve 412 was produced by applying a safety margin multiplier of 2.0 to the strength-duration curve 402, i.e., by multiplying by 2.0 each pulse amplitude at which capture occurs for each of a plurality of possible pulse widths within a specified range (e.g., from 0.2 msec to 1.5 msec, but not limited thereto). As noted above, instead of a safety margin being a multiplier, the safety margin can be a value that is additive, e.g., in the range of 1.0 to 3.0 V, but is not limited thereto. For example, if an additive safety margin is 1.5 V, then an iso-safety factor strength duration curve can be produced by adding 1.5 V to each pulse amplitude at which capture occurs for each of a plurality of possible pulse widths within a specified range (e.g., from 0.2 msec to 1.5 msec, but not limited thereto).

As will be appreciated from the following discussion, in accordance with certain embodiments, an iso-safety factor strength duration curve (e.g., 412) (which can be the information indicative thereof) and a pacing current drain curve (which can be the information indicative thereof) are produced and used to identify a preferred pacing parameter set that includes a preferred pulse amplitude and a preferred pulse width for pacing pulses, which set substantially minimizes current drain (and thereby charge drain) from the battery of an IMD per pacing pulse that is delivered while maintaining an equivalent safety margin specified by a clinician, or alternatively by default.

FIG. 4B is a graph that illustrates an example pacing current drain curve 422, wherein the graph has pacing pulse width plotted on the horizontal axis and pacing current drain (la) plotted on the vertical axis. The example pacing current drain curve 422 is a representation of the relationship between current drain (I_{d}) and pulse width for an IMD capable of delivering pacing pulses to a patient. As can be appreciated from FIG. 4B, the minimum amount of current drain for the example pacing current drain curve 422 occurs when the pulse width is about 0.5 msec.

FIG. 4C is a graph that includes the example strength duration curve 402 and the example iso-safety factor strength duration curve 412 (introduced in FIG. 4A), as well as the example pacing current drain curve 422 (introduced in FIG. 4B). As can be appreciated from FIG. 4C, since the minimum current drain occurs when the pulse width is 0.5 msec, and the capture threshold with the safety margin applied thereto is equal to 1.25 V when the pulse width is 0.5 msec, the curves 412 and 422 (and more specifically, the information indicative thereof) can be used to identify a preferred (aka recommended) pacing parameter set (corresponding to the point labeled 426) that includes a preferred (aka recommended) pulse amplitude of 1.25 V and a preferred (aka recommended) pulse width of 0.5 msec.

One way to generate an iso-safety factor strength duration curve (similar to 412) for a patient would be to determine corresponding capture thresholds for all possible pulse widths within a specified range (e.g., from 0.1 msec to 1.5 msec), to produce a strength duration curve (similar to 402), and then apply a safety margin (aka safety factor) to the determined capture thresholds (with the safety margin applied). However, producing an iso-safety factor strength duration curve (and more specifically, information indicative thereof) by measuring a capture threshold for all possible pulse widths within a specified range would be very time consuming and not practical, e.g., during a patient follow-up visit. In according with certain embodiments of the present technology, rather than measuring a capture threshold for a patient at all possible pulse widths within a specified range, a capture threshold is measured at a single pulse width that is specified by a clinician (or by default), and then a safety margin is applied by the clinician (or automatically by default), wherein the safety margin can be specified by the clinician (or by default). Then, based on the capture threshold with the safety margin applied at the single pulse width, modeling is used to produce an iso-safety factor strength duration curve that is tailored to the specific patient and the specific IMD. As will be described in additional detail below, in accordance with certain embodiments of the present technology, such modeling is based on modeling equations and patient population data uploaded from actual pacemakers used to treat patients.

In according with other embodiments of the present technology, rather than measuring a capture threshold for a patient at all possible pulse widths within a specified range, capture thresholds are measured at a few (e.g., two to five) pulse widths that are specified by a clinician (or by default), and then a safety margin is applied by the clinician (or automatically by default) to the capture thresholds, wherein the safety margin can be specified by the clinician (or by default). Then, based on the capture thresholds with the safety margin applied at the few pulse widths, modeling is used to produce an iso-safety factor strength duration curve that is tailored to the specific patient and the specific IMD, wherein such modeling is based on modeling equations and patient population data uploaded from actual pacemakers used to treat patients.

Delivering pacing pulses at a capture threshold determined for a patient does not provide acceptable safety tolerance for possible fluctuations in capture threshold levels resulting from the acute to chronic stabilization or periodic metabolic changes. For instance, eating, sleeping, insulin, sodium or potassium infusion, antiarrhythmic drugs have been shown to increase capture thresholds. Accordingly, once a capture threshold has been measured for a patient, an adequate safety margin (aka safety factor) should be applied. Clinicians often have their own preferred practices and procedures for determining a pacing parameter set that provides what the physician believes to be an adequate safety margin (aka safety factor) for their patients. The safety margin is used to prevent capture from being lost due to variation in a patient's capture threshold. For example, the safety margin is designed to take into account the circadian variations in a patient's capture threshold which is influenced in variable manner from one patient to another and within an individual patient by sleep, meals, physical activity, etc., as noted above. Some clinicians may use a higher safety margin for patients that are pacemaker-dependent patients, compared to the safety margin that the clinicians use for patients that are not pacemaker-dependent. Some clinicians may specify the safety margin as a multiplier, e.g., in the range of 1.2 to 2.0, but not limited thereto. For example, if the safety margin is specified using a multiplier of 1.4, then the pulse amplitude that is used for delivering pacing pulses, having a specified pulse width, would be 1.4 times the pulse amplitude at the capture threshold corresponding to the specified pulse width. As explained above, using multiplier safety martin of 1.4 effectively doubles (i.e., increases by 100%) the stimulation energy, since 1.4\hat{ }2≈2. Other clinicians may specify a safety margin that is additive, e.g., in the range of 1.0 to 3.0 Volts, but not limited thereto. For example, if an additive safety margin is specified as 1.5 Volts, then the pulse amplitude that is used for delivering pacing pulses, having a specified pulse width, would 1.5 Volts greater than the capture threshold corresponding to the specified pulse width.

As will be described in additional detail below, certain embodiments of the present technology use a clinician's selected set of pacing parameters (aka a clinician selected pacing parameter set) which already included consideration for a prescribed safety margin to generate an iso-safety factor strength duration curve for each of a plurality of pulse widths (aka stimulus durations) within a specified range, e.g., from 0.1 msec to 1.5 msec, but not limited thereto, at 0.1 msec increments, or some other increments. Once the iso-safety factor strength duration curve is generated, the current drain for each of a plurality of pacing parameter sets along the iso-safety factor strength duration curve is determined to thereby also generate a current drain curve. Based on the iso-safety factor strength duration curve and the current drain curve, a preferred pacing parameter set is recommended that has a lower current drain and equivalent safety margin as the pacing parameter set that had been selected by the clinician. As the phrase is used herein, a pacing parameter set includes a pulse amplitude and a pulse width. It is also noted that the phrase pacing parameter set can alternatively be referred to as (i.e., is also known as (aka)) a set of pacing parameters. Further, it is noted that the phrase “based on” as used throughout this description means “based at least in part on,” meaning that a determination that is based on something may also be based on one or more additional factors, unless specified otherwise.

In accordance with certain embodiments, when an iso-strength duration curve is generated (aka determined or produced) for a patient, it is generated based on a simplified derivation of maximum membrane response voltage equations first introduced by H. A. Blair in their articles “On the intensity-time relations for stimulation by electric currents—I,” J. Gen. Physiol., vol. 15, pp. 709-729, 1932; and “On the intensity-time relations for stimulation by electric currents—II,” J. Gen. Physiol., vol. 15, pp. 751-755, 1932, which are incorporated herein by reference. In those articles, Blair showed that tissue can be modelled by a simple resistor-capacitor (RC) network, and to trigger excitation the transmembrane potential of a cell needs to be reduced by a critical amount. Beneficially, use of models introduced by Blair tolerate a decaying exponential pacing pulse properties that are used by modern pacemakers, such as LPs, which is not the case for earlier developed modeling algorithms that had been by L. Lapicque in 1909, and by G. Weiss in 1901. Rather, with the earlier developed modeling algorithms that had been introduced by L. Lapicque in 1909, and by G. Weiss in 1901, use of such algorithms for modelling decaying exponential pacing pulse properties that are used by modern pacemakers requires a conversion from a decaying exponential to constant current and back to use the associated equations to estimate a strength duration curve, which introduces errors particularly at long pulse widths when capture has been achieved at the rheobase.

In accordance with certain embodiments, the membrane response, Vₘₑₘ(t), is described as a function of pacing amplitude (PA) using the following equation:

$${V}_{mem} ( t ) = PA \cdot [ \frac{1}{[ \frac{1}{{τ}_{m}} ] - [ \frac{1}{( {R}_{load} \cdot {C}_{pace} )} ]} ] \cdot ( exp [ \frac{- ( t )}{( {R}_{load} \cdot {C}_{pace} )} ] - exp [ \frac{- ( t )}{( {τ}_{m} )} ] ) \qquad ( EQ 1 )$$

where,
Vₘₑₘ(t) is the membrane response,
PA is the pulse amplitude (which is programmable),
τₘ is the membrane time constant (which is derived),
R_{load} is the pacing impedance (which is measured),
C_{pace} is the capacitance of the pace capacitor (which is constant), and
t is the stimulus duration, aka the pulse width (which is programmable).

The stimulus duration with the maximum membrane response (Tₘₐₓ) for an infinitely long stimulus duration can be determined by taking the derivative of the above equation (i.e., taking the derivative of EQ2), which results in the following equation:

$${T}_{max} = Ln [ \frac{[ \frac{{τ}_{m}}{( {R}_{load} \cdot {C}_{pace} )} ]}{( [ \frac{1}{( {R}_{load} \cdot {C}_{pace} )} ] - [ \frac{1}{( {τ}_{m} )} ] )} ] \qquad ( EQ2 )$$

where,
Tₘₐₓ is stimulus duration (aka pulse with) with the maximum membrane response,
Ln is the natural log,
τₘ is the membrane time constant (which is derived),
R_{load} is the pacing impedance (which is measured), and
C_{pace} is the capacitance of the pace capacitor (which is constant).

The above equation EQ2 is useful to determine the maximum membrane response required for stimulation when a stimulation pulse width (aka stimulus duration) is beyond rheobase. Explained another way, Tₘₐₓ is the stimulus duration (aka pulse width) needed for the membrane response voltage (Vₘₑₘ) to reach its maximum value for an infinitely long stimulus duration, which can be referred to as the maximum membrane response (V_{mem_max}). In accordance with certain embodiments, to generate an iso-safety factor strength duration curve (similar to 422 in FIGS. 4B and 4C), the maximum membrane response (Vₘₑₘ) is calculated from the above described equation EQ1 using a selected pacing amplitude (PA), a selected stimulation duration (t) (which can also be referred to as a selected pulse width), the membrane time constant (τₘ), the measured pacing impedance (R_{load}), and the pacing capacitance (C_{pace}). If a selected pacing duration (t) (aka pulse width) is greater than the Tₘₐₓ (i.e., if t>Tₘₐₓ), then Tₘₐₓ is used as the stimulus duration (t) in the equation EQ1 above, when determining the membrane response Vₘₑₘ(t). If the selected pacing duration (t) (aka pulse width) is less than Tₘₐₓ (i.e., if t<Tₘₐₓ), then the selected pacing duration is used as the stimulus duration (t) in the equation EQ1 above, when determining the membrane response Vₘₑₘ(t). Accordingly, the maximum membrane response (Vₘₑₘ) can be determined using the below equation EQ3, which is based on EQ1 introduced above.

$${V}_{mem - max} ( t ) = PA \cdot [ \frac{1}{[ \frac{1}{{τ}_{m}} ] - [ \frac{1}{( {R}_{load} \cdot {C}_{pace} )} ]} ] \cdot ( exp [ \frac{- ( t )}{( {R}_{load} \cdot {C}_{pace} )} ] - exp [ \frac{- ( t )}{( {τ}_{m} )} ] ) \qquad ( EQ3 )$$

where,
V_{mem_max}(t) is the maximum membrane response,
PA is the pulse amplitude (which is programmable) at the test pulse width t,
τₘ is the membrane time constant (which is derived),
R_{load} is the pacing impedance (which is measured),
C_{pace} is the capacitance of the pace capacitor (which is constant), and
t is the test stimulus duration, aka the test pulse width (which is programmable) (where t=Tₘₐₓ (if t>Tₘₐₓ), or t=t (if t<Tₘₐₓ)).

In accordance with certain embodiments, once the maximum membrane response value (V_{mem_max}) is determined, then pacing amplitudes to include the iso-safety factor strength duration curve are solved for algebraically, for each of a plurality of pulse widths (aka stimulus durations) within a specified range (e.g., from 0.1 msec to 1.5 msec, but not limited thereto, at increments of 0.1 msec, or some other increments), using the following equation:

$${PA}_{Estimated per pw} = {V}_{mem \_ max} \cdot [ \frac{[ \frac{1}{{τ}_{m}} ] - [ \frac{1}{( {R}_{load} \cdot {C}_{pace} )} ]}{( exp [ \frac{- ( t )}{( {R}_{load} \cdot {C}_{pace} )} ] - exp [ \frac{- ( t )}{( {τ}_{m} )} ] )} ] \qquad ( EQ4 )$$

where,
PA_{Estimated per pw} is a pulse amplitude calculated, for inclusion in the iso-safety factor strength duration curve that is being generated, for each of a plurality of pulse widths (aka stimulus durations) within a specified range (e.g., from 0.1 msec to 1.5 msec, but not limited thereto, at increments of 0.1 msec, but not limited thereto),
V_{mem_max} the maximum membrane response calculated using EQ3,
τₘ is the membrane time constant (which is derived),
R_{load} is the pacing impedance (which is measured),
C_{pace} is the capacitance of the pace capacitor (which is constant), and
t is the stimulus duration, aka the pacing pulse width (PW_{pace}) (which is programmable), for which the PA_{Estimated per pw} is being calculated.

In accordance with certain embodiments, as can be appreciated from the above discussed equations, a membrane time constant (τₘ) is used to model the iso-safety factor strength duration curve. In specific embodiments, the value for the membrane time constant (τₘ) is determined using empirical data and linear least squares regression methods with patient measured strength duration curves collected from LPs (or other types of IMDs) implanted in patients.

As noted above, in order to identify the preferred pacing parameter set, in addition to determining the iso-safety factor strength duration curve, the pacing current drain curve should also be determined. In accordance with certain embodiments, the current drain curve can be determined using the below equation:

$${I}_{pace} = {Q}_{pace} \cdot \frac{Average Base Rate}{60} \qquad ( EQ5 )$$

where,
I_{pace} is the pacing current, aka pacing current drain (I_{d}) (which is derived),
Q_{pace} is the pacing charge, aka pacing charge drain (which is derived using EQ6 and EQ7, below), and
Average Base Rate is the average base pacing rate per minute (e.g., 60 beats per minute (bpm)).

The following equation can be used to determine the pacing charge, aka the pacing charge drain:

$${Q}_{pace} = M \cdot PQ \qquad ( EQ6 )$$

where,
Q_{pace} is the pacing charge, aka pacing charge drain (which is calculated),
M is the charge multiplier (which is one of a plurality of possible constants, e.g., 0.5, 1.0, 1.5, 2.0, or 3.0), and
PQ is the base pacing charge (which is derived using EQ7 below).

The value of the charge multiplier M in EQ6 depends on the magnitude of the pulse amplitude (PA), for the pacing charge (Q_{pace}). For the following example it is assumed that a charge pump being used is capable of operating as a halfer. If PA<=2 V, then the value of M=0.5; if 2<PA<=3, then value of the M=1.0; if 3<PA<=4, then value of the M=1.5; if 4<PA<=5.0, then value of the M=2.0; and if PA>5, then value of the M=3.0. This is just one example of how the charge multiplier M, which can also be referred to as a multiplier factor, can be used. Other variations are also possible and within the embodiments described herein.

The base pacing charge (PQ) can be determined using the following equation:

$$PQ = {C}_{pace} \cdot PA \cdot ( 1 - exp [ \frac{- ( {PW}_{pace} )}{( {R}_{load} \cdot {C}_{pace} )} ] ) \qquad ( EQ7 )$$

where,
C_{pace} is the capacitance of the pace capacitor (which is constant), and
PA is the pulse amplitude (which is programmable),
R_{load} is the pacing impedance (which is measured),
PW_{pace} is the pacing pulse width (which is programmable).

Combining EQ5, EQ6 and EQ7, the current drain curve can be determined using the below equation:

$${I}_{pace} = M \cdot {C}_{pace} \cdot PA \cdot ( 1 - exp [ \frac{- ( {PW}_{pace} )}{( {R}_{load} \cdot {C}_{pace} )} ] ) \cdot \frac{Average Base Rate}{60} \qquad ( EQ8 )$$

where,
I_{pace} is the pacing current, aka pacing current drain (which is derived),
M is the charge multiplier (which is one of a plurality of possible constants, e.g., 0.5, 1.0, 1.5, 2.0, or 3.0),
C_{pace} is the capacitance of the pace capacitor (which is constant), and
PA is the pulse amplitude (which is programmable),
PW_{pace} is the pacing pulse width (which is programmable), and
Average Base Rate is the average based pacing rate per minute (e.g., 60 beats per minute).

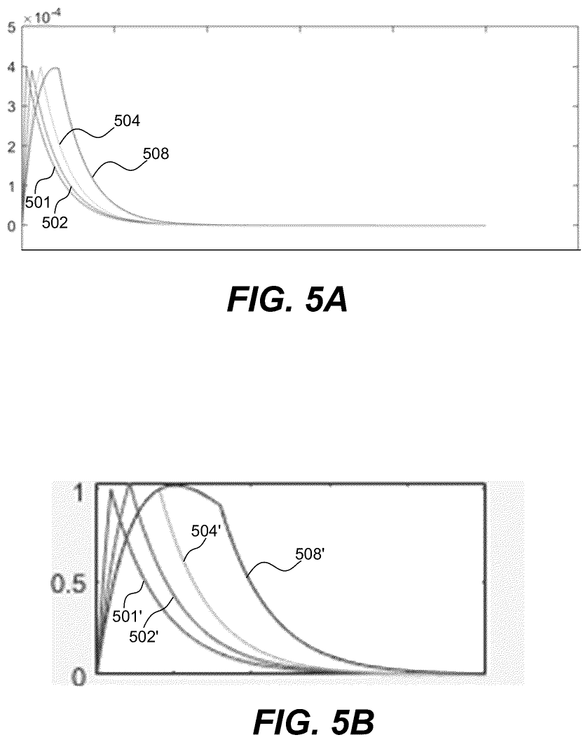

FIG. 5A is a graph that shows a representation of the membrane responses Vₘₑₘ(t), which can be modeled using the equation EQ1 above. More specifically, the curves 501, 502, 504, and 508, respectively, are membrane responses for capture thresholds at pulse widths of 0.1 msec, 0.2 msec, 0.4 msec, and 0.8 msec. The units are somewhat arbitrary but normally, with a given pacing amplitude and pulse width, when the capture threshold is reached, regardless of pulse amplitude each captured pace comes to the same membrane response. FIG. 5B includes normalized versions of the membrane response curves introduced in FIG. 5A, with the curve 501′ being a normalized version of the curve 501, the curve 502′ being a normalized version of the curve 502, etc. Occasionally, as can be appreciated from FIG. 5B, when a pacing pulse is delivered for a long period of time (i.e., has a long stimulation duration, aka pulse width), the membrane response will surpass the peak, which is known as “over shocking.” Over shocking the membrane results in wasted energy since the capture threshold for stimulation was reached at the peak.

With respect to equations EQ1 and EQ3 discussed above, in certain embodiments the pulse amplitude (PA) in those equations is the capture threshold at a specified stimulus duration t (aka a specified pulse width, e.g., a first pulse width). In alternative embodiments, the PA in equations EQ1 and EQ3 is a pacing amplitude equal to the capture threshold to which a multiplier or additive safety margin has been applied. In other words, in such alternative embodiments, the PA in equations EQ1 and EQ3 is equal to the product of the capture threshold (at the specified pulse width) multiplied by a multiplier type safety margin, or equal to the sum of the capture threshold (at the specified pulse width) plus an additive type safety margin.

With respect to equation EQ4 discussed above, in embodiments where the PA (in equations EQ1 and EQ3) is the capture threshold, then equation EQ4 can be used to determine the PA_{Estimated per pw} for additional pulse withs to thereby determine a strength duration curve. A specified safety margin can them be applied to the strength duration curve to determine the iso-factor strength duration curve. The specified safety margin can be multiplier type of safety margin, or an additive type of safety margin, as was described above.

With respect to EQ4 discussed above, in the alternative embodiments where the PA (in equations EQ1 and EQ3) is a pacing amplitude equal to the capture threshold to which a multiplier or additive type safety margin has already been applied, then equation EQ4 can be used to determine the PA_{Estimated per pw} for additional pulse withs to thereby directly determine the iso-factor strength duration curve.

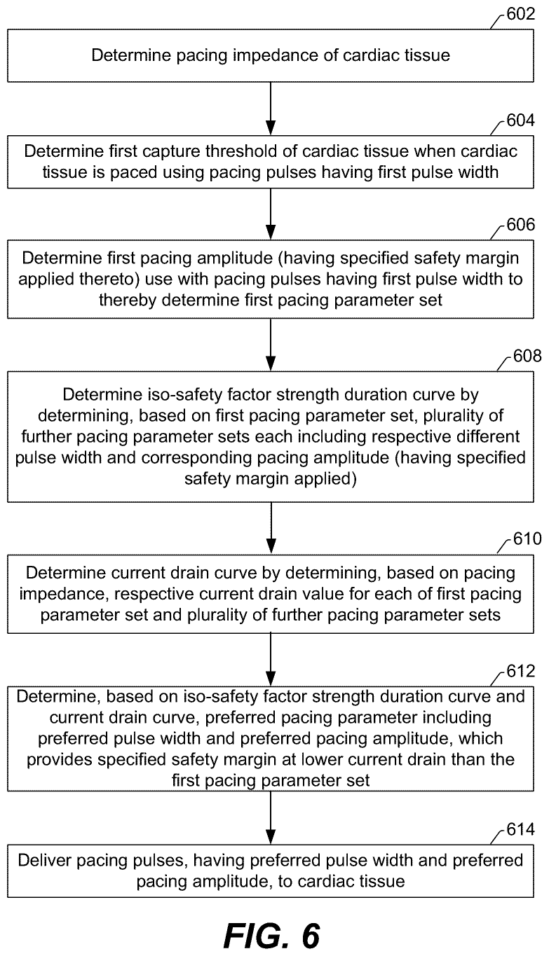

FIG. 6A is a high level flow diagram that is used to summarize methods according to certain embodiments of the present technology, which can be used to determine a preferred pacing parameter set for use in delivering pacing pulses to cardiac tissue. Referring to FIG. 6A, step 602 involves determining a pacing impedance of the cardiac tissue that is to be paced by the IMD. It is well known how an IMD that delivers pacing pulses can measure pacing impedance, and thus, further details of this step need not be described. Step 604 involves determining a first capture threshold of the cardiac tissue that is to be paced by the IMD when the cardiac tissue is paced using pacing pulses having a first pulse width. The first pulse width can be predetermined, e.g., a default pulse width. It would also be possible for the first pulse width to be specified by a user, via a user interface. It is well known how an IMD the delivers pacing pulses can determine a capture threshold for pacing pulses having a specified pulse width. For example, a capture threshold can be determined for a specific pulse width by incrementally increasing and/or decreasing pulse amplitudes of pacing pulses having the specific pulse width over a plurality of cardiac cycles and detecting, based on a sensed electrogram (EGM), when there is a transition from there being no capture to there being capture, and/or when there is a transition from there being capture to there being no capture. There are numerous ways in which steps 602 and 604 can be performed, which are well known, and embodiments of the present technology are not limited to any specific ways of performing steps 602 and 604.

Still referring to FIG. 6A, step 606 involves determining a first pacing amplitude having a specified safety margin applied, which can be used by the IMD to deliver pacing pulses having the first pulse width to the cardiac tissue at the specified safety margin above the first capture threshold. More generally, step 606 involves determining a first pacing parameter set, where the first pulse width and the first pacing amplitude (having the specified safety margin applied) collectively comprise the first pacing parameter set. Step 606 can be performed by determining a product of the specified safety margin and the capture threshold, wherein the specified safety margin is predetermined and is a multiplier type of safety margin. Alternatively, step 606 can be performed by determining a sum of the specified safety margin and the capture threshold, wherein the specified safety margin is predetermined and is an additive type of safety margin. In another embodiment, step 606 is performed by accepting, from a user via a user interface, the first pacing amplitude having the specified safety margin already applied, as determined by the user. In still another embodiments, step 606 is performed by accepting, from a user via a user interface, the specified safety margin, and determining a product of the specified safety margin and the capture threshold. In a further embodiment, step 606 is performed by accepting, from a user via a user interface, the specified safety margin, and determining a sum of the specified safety margin and the capture threshold. Other variations are possible and within the scope of the embodiments described herein.

Step 608 involves determining an estimate of a maximum membrane response for the cardiac tissue of the patient's heart, based on the pacing impedance, the first pulse width, and the first pacing amplitude having the specified safety margin applied, e.g., using the equation EQ3 discussed above.

Step 610 involves determining an iso-safety factor strength duration curve by determining, based on the first pacing parameter set, a plurality of further pacing parameter sets that each includes a respective different pulse width and a corresponding pacing amplitude having the specified safety margin applied. Determining the iso-safety factor strength duration curve at step 610 refers to determining the data indicative thereof (i.e., values for multiple points along the curve), but not necessarily determining a graphical representation of the curve, although that is also possible if the curve is to be displayed to a user via a user interface. In accordance with certain embodiments, the iso-safety factor strength duration curve is determined at step 610 without determining any additional capture threshold, besides the first capture threshold determined at step 604. In specific embodiments, step 610 is performed using the estimate of the maximum membrane response for the cardiac tissue of the patient's heart, determined at step 608 based on the pacing impedance, the first pulse width, and the first pacing amplitude having the specified safety margin applied, e.g., using the equation EQ3 discussed above. Then, for each of the further pacing parameter sets, determining the corresponding pacing pulse amplitude to use with the respective pulse width, based on the estimate of the maximum membrane response, the respective pulse width, and the pacing impedance, e.g., using the equation EQ4 discussed above. In other embodiments, one or more additional capture thresholds are also determined for one or more additional pulse widths, and the additional capture threshold(s) is/are also used to produce the iso-safety factor strength duration curve. While steps 608 and 610 are shown as being separately performed in FIG. 6A, it is also within the scope of the embodiments described herein that step 608 is performed as part of step 610, i.e., that steps 608 and 610 are sub-steps of a same step.

In certain embodiments, where one or more additional capture thresholds are also determined for one or more additional pulse widths, a patient specific membrane time constant (τₘ) is determined based on the multiple capture thresholds with the specified safety margin applied, and the patient specific membrane time constant (τₘ) is used to produce iso-safety factor strength duration curve, rather than using a membrane time constant (τₘ) that is based on empirical patient population data. This can include performing error minimization using the Levenberg-Marquart (L-M) nonlinear least squares regression method. The L-M nonlinear least squares regression method initializes unknown parameters for a given mathematical expression, and iteratively updates until a resulting residual sum of squares is minimized. In this case, using the equation EQ4 discussed above, values for the maximum membrane response (V_{mem_max}) and the membrane time constant (τₘ) may be iteratively replaced to minimize the sum of squared error. The results are values for the maximum membrane response (V_{mem_max}) values that converge to near equivalent values with the optimal membrane time constant (τₘ) regardless of pulse width and pulse amplitude from the measured capture threshold values. Alternatively, a less computationally intensive way to determine a patient specific membrane time constant (τₘ) involves sweeping possible values for the patient specific membrane time constant (τₘ) within a range (e.g., from 0.1 msec to 1.0 msec, but not limited thereto) to find a value that minimizes error in EQ4. Once the patient specific membrane time constant (τₘ) is calculated it may be used, rather than a membrane time constant (τₘ) that is based on empirical patient population data, to determine the preferred pacing parameter set.

Step 612 involves determining a current drain curve by determining, based on the pacing impedance, a respective current drain value for each the first pacing parameter set and the plurality of further pacing parameter sets. Determining the current drain curve at step 612 refers to determining the data indicative thereof (i.e., values for multiple points along the curve) but not necessarily determining the graphical representation of the curve, although that is also possible if the curve is to be displayed to a user via a user interface.

Step 614 involves determining, based on the iso-safety factor strength duration curve and the current drain curve, a preferred pacing parameter set that includes a preferred pulse width and a preferred pacing amplitude, which provides the specified safety margin at a lower current drain than the first pacing parameter set, thereby increasing the longevity of the IMD compared to if the first pacing parameter set were to be used for pacing. Preferably, the preferred pacing parameter set is selected such that it corresponds to a minimum current drain, in order to maximize the longevity of the IMD.

In certain embodiments, the preferred pacing parameter set is determined at step 614 by an external device (e.g., 109) that is configured to communicate with the IMD. In such an embodiment, it is likely that at least some of the other steps described with reference to FIG. 6A are also performed by, or at least under the control of, the same external device. In other embodiments, the determining of the preferred pacing parameter set at step 614 is performed by the IMD itself, by one or more processors thereof. In other words, processor(s) of the IMD can determine the preferred pacing parameter set, then the processor(s) can control the IMD to perform pacing using the preferred pacing parameter set. In accordance with certain embodiments, the estimate of the maximum membrane response is also determined based on a membrane time constant (τₘ) that is determined based on empirical patient population data.

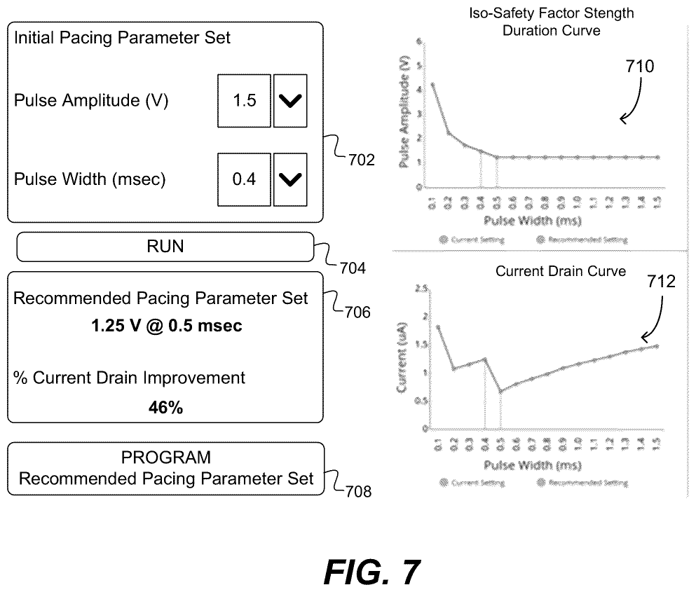

Step 616 involves an IMD delivering pacing pulses, having the preferred pulse width and the preferred pacing amplitude, to the cardiac tissue of the patient's heart. Where the IMD performs step 614, the IMD can perform step 616 following step 614. Alternatively, as will be appreciated from FIG. 7 discussed below, information indicative of the preferred pacing parameter set (that includes the preferred pulse width and the preferred pacing amplitude) can be displayed to a user via a user interface of an external device that is configured to communicate with the IMD. Thereafter, the IMD can be programmed by the external device (or another external device) to use the preferred pacing parameter set in response to the user accepting the preferred pacing parameter set via the user interface.

It is often the case that an IMD includes a charge pump that can apply one of a plurality of multiplier factors to a battery voltage, output by a battery of the IMD, to achieve a range of pacing amplitudes. In accordance with certain embodiments, the current drain curve determined at step 612 accounts for the plurality of multiplier factors and causes the current or charge drain curve to have multiple inflection points. Such multiplier factors are represented in equations EQ6 and EQ8, discussed above, by M.

In accordance with certain embodiments, rather than determining a current drain curve at step 612, and using the current drain curve at step 614 to determine the preferred pacing parameter set, a charge drain curve can be determined at step 612 and used at step 614 to determine the preferred pacing parameter set. Such a charge drain curve would have the same morphology as the current drain curve, but would specify an amount of charge drained per pacing pulse in coulombs (rather current drained per pacing pulse in amps) at each of a plurality of different pulse widths and pulse amplitudes. Regardless of whether a current drain curve or a charge drain curve is determined and used, the pulse width corresponding to where the current drain or charge drain is lowest can be used to determine the preferred pacing parameter set.

FIG. 6B is a high level flow diagram that is used to summarize methods according to alternative embodiments of the present technology, which can be used to determine a preferred pacing parameter set for use in delivering pacing pulses to cardiac tissue. Steps 602 and 604 in FIG. 6B are the same as steps 602 and 604 in FIG. 6A, and thus, need not be described again. Step 605 in FIG. 6B involves determining an estimate of a maximum membrane response for cardiac tissue of the patient's heart based on the pacing impedance (determined at step 602), the first pulse width, and the first capture threshold (determined at step 604), e.g., using equation EQ3 discussed above. In the embodiment of FIG. 6B, the first pulse width and the first capture threshold can be collectively referred to as a first pacing parameter set. The determination of the estimate of the maximum membrane response based on the pacing impedance (determined at step 602), the first pulse width, and the first capture threshold at step 605, is in contrast to step 604 (in FIG. 6A) where the estimate of the maximum membrane response was based on the pacing impedance (determined at step 602), the first pulse width, and the first pacing amplitude (having the specified safety margin applied, determined at step 606). In other words, step 605 uses the capture threshold (without a safety margin applied thereto) to estimate the maximum membrane response, whereas step 604 instead used the first pacing amplitude (having the safety margin applied) to estimate the maximum membrane response, wherein the first pacing amplitude was a product of the specified safety margin and the capture threshold (if the specified safety margin was a multiplier type of safety margin), or the first pacing amplitude was a sum of the specified safety margin and the capture threshold (if the specified safety margin was an additive type of safety margin). More specifically, in the embodiment of FIG. 6B, the pulse amplitude (PA) used in the equation EQ3 is the capture threshold. By contrast, in the embodiment of FIG. 6A, the PA used in equation EQ3 was the first pacing amplitude having the specified safety margin applied. However, as was also the case in the embodiment of FIG. 6A, the first pulse width and the capture threshold with the specified safety margin applied, collectively comprise a first pacing parameter set, relative to which a preferred pacing parameter set is later determined, at step 614, which provides the specified safety margin at a lower current or charge drain than the first pacing parameter set.

Step 607 in FIG. 6B involves determining a strength duration curve by determining, based on the capture threshold (determined at step 604) and the estimate of the maximum membrane response (determined at step 605), a plurality of further pacing parameter sets each including respective different pulse width and corresponding pacing amplitude. Determining the strength duration curve at step 607 refers to determining the data indicative thereof (i.e., values for multiple points along the curve), but not necessarily determining a graphical representation of the curve, although that is also possible if the curve is to be displayed to a user via a user interface. In certain embodiments, the equation EQ4 discussed above is used to determine the PA_{Estimated per pw} for additional pulse widths to thereby determine the strength duration curve.

In accordance with certain embodiments, the strength duration curve is determined at step 607 without determining any additional capture threshold, besides the first capture threshold determined at step 604. Alternatively, one or more additional capture thresholds are also determined for one or more additional pulse widths, a patient specific membrane time constant (τₘ) is determined based on the multiple capture thresholds (without a specified safety margin applied), and the patient specific membrane time constant (τₘ) is used to produce the strength duration curve, rather than using a membrane time constant (τₘ) that is based on empirical patient population data.

Step 609 in FIG. 6B involves determining an iso-safety factor strength duration curve by applying a specified safety margin to the strength duration curve determined at step 607. Determining the iso-safety factor strength duration curve at step 609 refers to determining the data indicative thereof (i.e., values for multiple points along the curve), but not necessarily determining a graphical representation of the curve, although that is also possible if the curve is to be displayed to a user via a user interface.

Step 609 can be performed by determining a product of the specified safety margin and each of a plurality of pacing pulse amplitude points on the strength duration curve (determined at step 607), if the specified safety margin is a multiplier type of safety margin. Alternatively, step 609 can be performed by determining a sum of the specified safety margin and each of a plurality of pacing pulse amplitude points on the strength duration curve (determined at step 607), if the specified safety margin is an additive type of safety margin. In certain embodiments, step 609 involves applying the safety margin to respective pacing amplitudes of the first pacing parameter set and the plurality of further pacing parameter sets to thereby determine further respective pacing amplitudes with the safety margin applied, which are included in the iso-safety factor strength duration curve.

In an embodiment, step 609 is performed in part by accepting, from a user via a user interface, the first capture threshold having the specified safety margin already applied, as determined by the user. In still another embodiments, step 609 is performed in part by accepting, from a user via a user interface, the specified safety margin, and determining a product of the specified safety margin and the capture threshold. In a further embodiment, step 609 is performed in part by accepting, from a user via a user interface, the specified safety margin, and determining a sum of the specified safety margin and the capture threshold. Other variations are possible and within the scope of the embodiments described herein.

Steps 610, 612, and 614 in FIG. 6B are the same as steps 610, 612, and 614 in FIG. 6A, and thus need not be described again.

As can be appreciated from the above discussion, in certain embodiments a membrane time constant (that is used to determine an estimate of a maximum membrane response) is determined based on a measured capture threshold and empirical patient population data. When determining the membrane time constant using empirical patient population data, there is an assumption that a patient for which the membrane time constant is being determined has a similar membrane time constant to those patients whose data is included in the empirical patient population data. However, that might not always be the case, as different patients may on rare occasions have significantly different (e.g., significantly longer or shorter) membrane time constants than the patients whose data is included in the empirical patient population data. This means that it is possible that an iso-safety factor strength duration curve generated using a membrane time constant determined based on empirical patient population data may not provide for a user specified (e.g., physician specified) safety margin. More specifically, it is possible that a preferred pulse width and preferred pacing amplitude determined at an instance of step 614, while providing for a lower current drain (than an initial or first pacing parameter set), may not achieve the specified safety margin. To account for this, in accordance with certain embodiments, after a preferred pulse width and a preferred pacing amplitude is identified at an instance of step 614, a test is performed to check whether the identified preferred pulse width and preferred pacing amplitude does indeed satisfy the specified safety margin. Such a test can be a manual test. Alternatively, such a test can be automated, similar to how automatic capture detection and/or automatic capture confirmation test can be performed, but is not limited thereto. Further, it is noted that it rare instances it is possible that a preferred pulse width and a preferred pacing amplitude is the same as the initial or first pacing parameter set. In other words, in rare cases the initial pacing parameter set that is specified by a user (or by default) may be the preferred pacing parameter set that provides for the specified safety margin and for the lowest current drain. In such cases, at an instance of step 614 there can be an indication that the preferred pacing parameter set is the same as the first pacing parameter set, or there can be a notification that a pacing parameter set that is preferred over the first pacing parameter set cannot be identified. Other variations are also possible and within the scope of the embodiments described herein.

The methods summarized above with reference to FIGS. 6A and 6B, can be used with any type of IMD that includes or is couplable to at least two electrodes that are used to deliver the pacing pulses to the cardiac tissue of a patient's heart. For example, the IMD can be an LP, example details of which were described above. Alternatively, the IMD can be a conventional pacemaker that is attached to one or more leads each of which include one or more electrodes. It would also be possible for the IMD to be an implantable cardioverter defibrillator (ICD) that includes pacing capabilities. Other variations are also possible and within the scope of the embodiments described herein.

When used with multiple LPs implanted in different cardiac chambers of a patient's heart, a method described herein can be performed for each of the LPs to thereby determine a different preferred pacing parameter set for each of the of LPs.

When used with a pacemaker to which one or more leads having electrode(s) is/are attached, such a method can be performed for each of a plurality of different electrode combinations to thereby determine a different preferred pacing parameter set for each of the plurality of different electrode combinations. The current drain (or charge drain) associate with the pacing parameter sets, along with various other factors, can be used to select which electrode combination should be used to pace a specific cardiac chamber, or to pace each of a plurality of different cardiac chambers.

In accordance with certain embodiments, a pacing amplitude (which can be the first capture threshold determined at step 604, or the first pacing amplitude having the specified safety margin applied determined at step 606) is converted to an average stimulation current using the below equation.

$${I}_{TH} = {I}_{AVE} = \frac{PA}{{PW}_{pace}} \cdot {C}_{pace} \cdot ( 1 - {exp}^{- ( \frac{{PW}_{pace}}{{R}_{load} \cdot {C}_{pace}} )} ) \qquad ( EQ9 )$$

C_{pace} is the capacitance of the pace capacitor (which is constant), and
PA is the pulse amplitude (which is programmable),
R_{load} is the pacing impedance (which is measured),
PW_{pace} is the pacing pulse width (which is programmable).

A rheobase with an assumed chronaxie is then solved for using the following equation:

$${Rheobase}_{test PW} = \frac{{I}_{AVE}}{( 1 + \frac{Chronaxie}{{PW}_{pace}} )} \qquad ( EQ10 )$$

The determined rheobase is then used to generate the strength duration curve and/or the iso-safety factor strength duration curve over a range of pulse widths. In certain embodiments, the chronaxie is determined based on empirical patient population data of strength duration curves, or a patient specific chronaxie can be determined based on multiple capture thresholds at margin corresponding to multiple tested pulse widths.

In the above equations EQ9 and EQ10, the estimates of the chronaxie and the rheobase are based on modeling algorithms developed by L. Lapicque in 1909, and by G. Weiss in 1901, and are thus in terms of constant current stimulation. Since modern pacemakers use decaying exponential voltage pulses (and thus, not constant current stimulation), there should be conversions between decaying exponentials, to constant current, then back to a peak voltage. This can be achieved using the below equations EQ11 through EQ19, which use the estimates of the rheobase and the chronaxie to determine an iso-safety factor strength duration curve including each of a plurality of pulse widths (aka stimulus durations) within a specified range (e.g., from 0.1 msec to 1.5 msec, but not limited thereto, at increments of 0.1 msec, or some other increments) and pacing amplitudes.

$${Q}_{Threshold per pw} = ( Rheobase \cdot Chronaxie ) + ( Rheobase \cdot PWpace ) \qquad ( EQ11 )$$

$${I}_{Average per pw} = \frac{{Q}_{Threshold per pw}}{PWpace} \qquad ( EQ12 )$$

$${I}_{Peak per pw} = \frac{{I}_{Average per pw}}{\frac{{R}_{load} \cdot {C}_{pace}}{{PW}_{pace}} ( 1 - {e}^{- ( \frac{{PW}_{pace}}{{R}_{load} \cdot {C}_{pace}} )} )} \qquad ( EQ13 )$$

$${P}_{PA per pw} = {R}_{load} \cdot {I}_{Peak per pw} \qquad ( EQ14 )$$

The pacing charge drain can then be solved for each combination of pulse width and pulse amplitude along the strength duration curve using the following equation:

$${P}_{PQ} = {P}_{PA} \cdot Cpace \cdot ( 1 - {e}^{- ( \frac{{PW}_{pace}}{{R}_{load} \cdot {C}_{pace}} )} ) \qquad ( EQ15 )$$

A charge pump multiplier M, e.g., such as 0.5, 1.0, 1.5, 2.0, or 3.0. which is a function of PPA is then determined and used to calculate the actual device pacing charge drain using the following equation:

$$Qpace = M \times {P}_{PQ} \qquad ( EQ16 )$$

Device pacing current drain is then calculated using the following equation, which assumes an average base pacing rate of 60 bpm, i.e., one beat per second or the actual average pacing rate derived from percentage pacing diagnostics:

$$Ipace = Qpace \cdot \frac{Average Base Rate}{60} \qquad ( EQ17 )$$

The preferred pacing parameter set can then be determined to be the pacing pulse width and pacing pulse amplitude associated with the lowest charge drain or current drain.

FIG. 7 is an example user interface of an external device that may be used to perform certain steps or functions of embodiments of the present technology described herein. Such an external device can be a programmer (e.g., 109) that is configured to communicate with one or more implanted IMDs (e.g., LPs). Such an external device can alternatively be a tablet computer, a laptop computer, a smartphone, and/or the like.

Referring to FIG. 7, shown in the upper left is an initial pacing parameter set 702 that can be specified at least in part by a user (e.g., clinician), wherein the initial pacing parameter set includes an initial pulse amplitude and an initial pulse width. In certain embodiments, the initial pacing amplitude already has the safety margin applied such that the initial pacing amplitude is at the specified safety margin above a capture threshold, wherein the specified safety margin may be a multiplier or additive, depending upon the specific implementation. In FIG. 7, the initial pulse amplitude is shown as being 1.5 V, and the initial pulse width is shown as being 0.4 msec. Such values can be selected by the user using, e.g., a keyboard, a drop down menu, or some other type of feature of a graphical user interface, but is not limited thereto.

After the initial pacing parameter set is specified, a user can select the run button 704, which initiates the determining of a preferred pacing parameter set 706 by one or more processors of the external device performing certain steps, e.g., steps 610, 612, and 614 in FIG. 6A. The preferred pacing parameter set 706, once determined, is displayed, as shown in FIG. 7. Additionally, a percentage current drain improvement of using the preferred pacing parameter set instead of the initial pacing parameter set can be calculated by the processor(s) and displayed to provide the user with an appreciation of how much better the preferred pacing parameter set is relative to the initial pacing parameter set. As shown in FIG. 7, a graphical representation of the iso-safety factor strength duration curve (e.g., 710) and a graphical representation of the current drain curve (e.g., 712) can optionally also be displayed to the user via the user interface of the external device. The user interface can also include a program button 708, which in response to being selected causes the IMD to be programmed to use the recommended pacing parameter set. Alternatively, the user can view the displayed recommended pacing parameter set, and manually enter the recommended pacing parameter set into another external device that is used to program the IMD. Other variations are also possible and within the scope of the embodiments described herein.

In accordance with certain embodiments, in additional using one of the implementations described above to determine a preferred pacing parameter set (including a preferred pulse amplitude and a preferred pulse width) and displaying that to a user via a user interface, e.g., as shown in FIG. 7, one or more additional pacing parameter sets that are less optimal (in terms of reducing current or charge drain) can also be determined and presented to the user, wherein the additional pacing parameter set(s) also result in lower current and charge drains than the initial pacing parameter set that was specified at least in part by the user. In other words, the preferred pacing parameter set and one or more additional pacing parameter sets can be presented to the user as possible options to use in place of the initial pacing parameter set, in order to improve device longevity, and then the user can choose which one of the options the user would like programmed into the IMD to be used for delivering pacing pulses. Such an embodiment can be useful, e.g., where a user (e.g., clinician) prefers to use pulse amplitudes and/or pulse widths that are within certain specific respective ranges.

As can be appreciated from the above discussion of FIGS. 1-3, pulses generated by a pulse generator of an IMD (e.g., LP), in addition to be used to produce pacing pulses, can also be used to produce conductive communication pulses for use in implant-to-implant (i2i) conductive communication between the IMD and another IMD, and/or for use in conductive communication between the IMD and an external device. As is the case with pacing pulses, conductive communication pulses also cause current and charge drain that depletes the battery of an IMD, and thus, it would also be beneficial to optimize parameters of conductive communication pulses for the purpose of increasing device longevity of an IMD. Certain embodiments of the present technology involve using similar techniques to those described above to select a preferred conductive communication parameter set that includes a preferred conductive communication pulse amplitude and/or a preferred conductive communication pulse width. Additionally, or alternatively, certain embodiments of the present technology involve using similar techniques to those described above to adjust the sensitivity of a conductive communication receiver of an IMD. In such embodiments, conductive communication parameters may be optimized automatically by an IMD, or a user can be presented with one or more options via a user interface of an external device and be given the opportunity to select which options provide for a sufficient level of communication quality while also conserving current and charge consumed from the battery of an IMD. Using such an embodiment, a preferred conductive communication parameter set can be determined for use in conductive communication between an IMD and another IMD, and a separate preferred conductive communication parameter set can be determined for use in conductive communication between the IMD and an external device. In certain embodiments, a user interface can show a user how adjusting certain conductive communication pulse parameters varies current and/or charge drain, to thereby enable the user to utilize such information to select pulse parameters to use for performing conductive communication. Similarly, a user can be able to adjust the sensitivity of a conductive communication receiver.

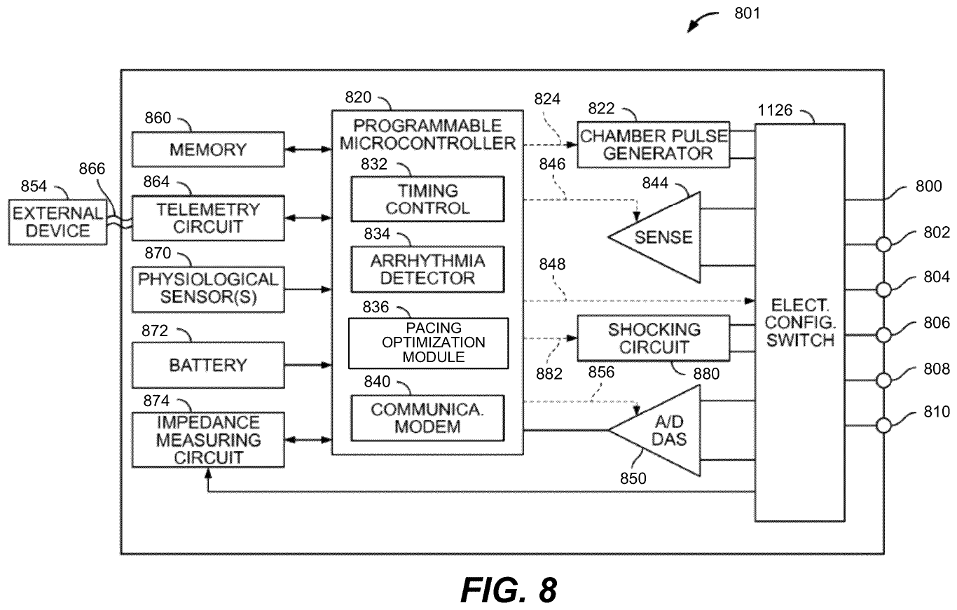

FIG. 8 shows a block diagram of one embodiment of an IMD (e.g., an LP or ICD) 801 that is implanted into the patient as part of the implantable cardiac system in accordance with certain embodiments herein. Optionally, the IMD 801 may provide full-function cardiac resynchronization therapy. Alternatively, the IMD 801 may be implemented with a reduced set of functions and components. For instance, the IMD may be implemented without ventricular sensing and pacing.

The IMD 801 has a housing 800 to hold the electronic/computing components. Housing 800 (which is often referred to as the “can”, “case”, “encasing”, or “case electrode”) may be programmably selected to act as the return electrode for certain stimulus modes. Housing 800 may further include a connector (not shown) with a plurality of terminals 802, 804, 806, 808, and 810. The terminals may be connected to electrodes that are located in various locations on housing 800 or elsewhere within and about the heart. The IMD 801 includes a programmable microcontroller 820 that controls various operations of the IMD 801, including cardiac monitoring and stimulation therapy. Microcontroller 820 includes a microprocessor (or equivalent control circuitry), RAM and/or ROM memory, logic and timing circuitry, state machine circuitry, and I/O circuitry.

The IMD 801 further includes a first pulse generator 822 that generates stimulation pulses for delivery by one or more electrodes coupled thereto. Pulse generator 822 is controlled by microcontroller 820 via control signal 824. Pulse generator 822 may be coupled to the select electrode(s) via an electrode configuration switch 826, which includes multiple switches for connecting the desired electrodes to the appropriate I/O circuits, thereby facilitating electrode programmability. Switch 826 is controlled by a control signal 828 from microcontroller 820.

In the embodiment of FIG. 8, a single pulse generator 822 is illustrated. Optionally, the IMD may include multiple pulse generators, similar to pulse generator 822, where each pulse generator is coupled to one or more electrodes and controlled by microcontroller 820 to deliver select stimulus pulse(s) to the corresponding one or more electrodes.

Microcontroller 820 is illustrated as including timing control circuitry 832 to control the timing of the stimulation pulses (e.g., pacing rate, atrio-ventricular (AV) delay, atrial interconduction (A-A) delay, or ventricular interconduction (V-V) delay, etc.). Timing control circuitry 832 may also be used for the timing of refractory periods, blanking intervals, noise detection windows, evoked response windows, alert intervals, marker channel timing, and so on. Microcontroller 820 also has an arrhythmia detector 834 for detecting arrhythmia conditions. Microcontroller 820 is also shown as including a pacing optimization module 836, which can be used for determining a preferred pacing parameter set using embodiments of the present technology described herein. Although not shown, the microcontroller 820 may further include other dedicated circuitry and/or firmware/software components that assist in monitoring various conditions of the patient's heart and managing pacing therapies.

The IMD 801 is further equipped with a communication modem (modulator/demodulator) 840 to enable wireless communication with other devices. Modem 840 may include one or more transmitters and two or more receivers as discussed herein in connection with FIG. 2. In one implementation, modem 840 may use low or high frequency modulation. As one example, modem 840 may transmit i2i messages and other signals through conductive communication between a pair of electrodes. The modem 840 can alternatively, or additionally, be used to provide RF communication and/or inductive communication. Modem 840 may be implemented in hardware as part of microcontroller 820, or as software/firmware instructions programmed into and executed by microcontroller 820. Alternatively, modem 840 may reside separately from the microcontroller as a standalone component.

The IMD 801 includes a sensing circuit 844 selectively coupled to one or more electrodes, that perform sensing operations, through switch 826 to detect the presence of cardiac activity in the right chambers of the heart. Sensing circuit 844 may include dedicated sense amplifiers, multiplexed amplifiers, or shared amplifiers. It may further employ one or more low power, precision amplifiers with programmable gain and/or automatic gain control, bandpass filtering, and threshold detection circuit to selectively sense the cardiac signal of interest. The automatic gain control enables the unit to sense low amplitude signal characteristics of atrial fibrillation. Switch 826 determines the sensing polarity of the cardiac signal by selectively closing the appropriate switches. In this way, the clinician may program the sensing polarity independent of the stimulation polarity.

The output of sensing circuit 844 is connected to microcontroller 820 which, in turn, triggers or inhibits the pulse generator 822 in response to the presence or absence of cardiac activity. Sensing circuit 844 receives a control signal 846 from microcontroller 820 for purposes of controlling the gain, threshold, polarization charge removal circuitry (not shown), and the timing of any blocking circuitry (not shown) coupled to the inputs of the sensing circuitry.

In the embodiment of FIG. 8, a single sensing circuit 844 is illustrated. Optionally, the IMD may include multiple sensing circuits, similar to sensing circuit 844, where each sensing circuit is coupled to one or more electrodes and controlled by microcontroller 820 to sense electrical activity detected at the corresponding one or more electrodes. Sensing circuit 844 may operate in a unipolar sensing configuration or in a bipolar sensing configuration.

The IMD 801 further includes an analog-to-digital (A/D) data acquisition system (DAS) 850 coupled to one or more electrodes via switch 826 to sample cardiac signals across any pair of desired electrodes. Data acquisition system 850 is configured to acquire intracardiac electrogram signals, convert the raw analog data into digital data, and store the digital data for later processing and/or telemetric transmission to an external device 109 (e.g., a programmer, local transceiver, or a diagnostic system analyzer). Data acquisition system 850 is controlled by a control signal 856 from the microcontroller 820.

The Microcontroller 820 is coupled to a memory 860 by a suitable data/address bus. The programmable operating parameters used by microcontroller 820 are stored in memory 860 and used to customize the operation of IMD 801 to suit the needs of a particular patient. Such operating parameters define, for example, pacing pulse amplitude, pulse duration, electrode polarity, rate, sensitivity (e.g., a sense detection threshold or gain), automatic features, arrhythmia detection criteria, and the amplitude, waveshape and vector of each shocking pulse to be delivered to the patient's heart within each respective tier of therapy.

The operating parameters of the IMD 801 may be non-invasively programmed into memory 860 through a telemetry circuit 864 in telemetric communication via communication link 866 with external device 109. Telemetry circuit 864 allows intracardiac electrograms and status information relating to the operation of IMD 801 (as contained in microcontroller 820 or memory 860) to be sent to external device 109 through communication link 866.

The IMD 801 can further include magnet detection circuitry (not shown), coupled to microcontroller 820, to detect when a magnet is placed over the unit. A clinician may use a magnet to perform various test functions of IMD 801 and/or to signal microcontroller 820 that external device 109 is in place to receive or transmit data to microcontroller 820 through telemetry circuits 864.

The IMD 801 can further include one or more physiological sensors 870. Such sensors are commonly referred to as “rate-responsive” sensors because they are typically used to adjust pacing stimulation rates according to the exercise state of the patient. However, physiological sensor 870 may further be used to detect changes in cardiac output, changes in the physiological condition of the heart, or diurnal changes in activity (e.g., detecting sleep and wake states). Signals generated by physiological sensors 870 are passed to microcontroller 820 for analysis. Microcontroller 820 responds by adjusting the various pacing parameters (such as rate, AV Delay, V-V Delay, etc.) at which the atrial and ventricular pacing pulses are administered. While shown as being included within IMD 801, physiological sensor(s) 870 may be external to IMD 801, yet still be implanted within or carried by the patient. Examples of physiologic sensors might include sensors that, for example, sense respiration rate, pH of blood, ventricular gradient, activity, position/posture, minute ventilation (MV), and so forth.

A battery 872 provides operating power to all of the components in IMD 801. Battery 872 is capable of operating at low current drains for long periods of time, and is capable of providing high-current pulses (for capacitor charging) when the patient requires a shock pulse (e.g., in excess of 2 A, at voltages above 2 V, for periods of 10 seconds or more). Battery 872 also desirably has a predictable discharge characteristic so that elective replacement time can be detected. As one example, IMD 801 employs lithium/silver vanadium oxide batteries.

The IMD 801 further includes an impedance measuring circuit 874, which can be used for many things, including: lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring stroke volume; and detecting the opening of heart valves; and so forth. Impedance measuring circuit 874 is coupled to switch 826 so that any desired electrode may be used. In this embodiment IMD 801 further includes a shocking circuit 880 coupled to microcontroller 820 by a data/address bus 882.

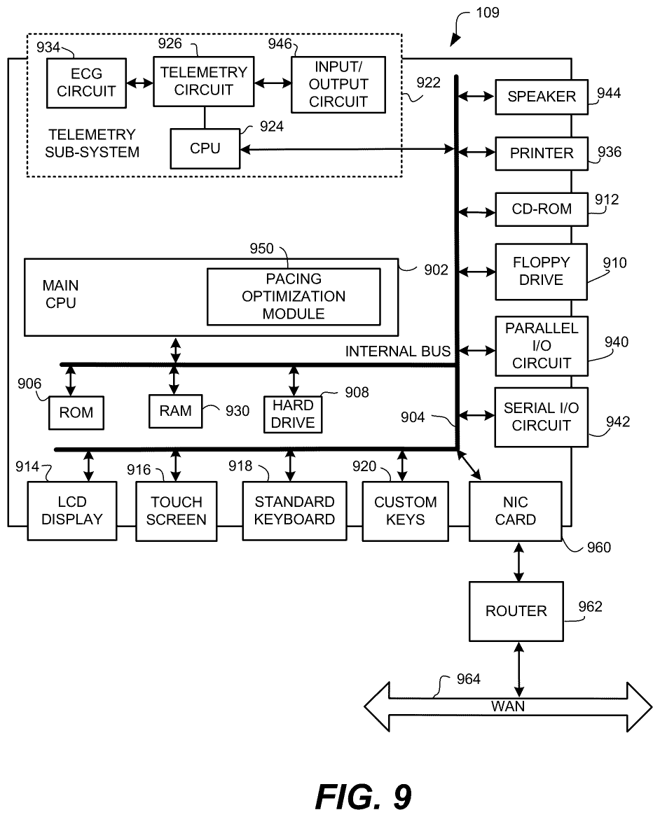

FIG. 9 illustrates example components of an example external device 109 for use in communicating with and/or programming the LPs 102, or other type of IMD. More generally, the external device 109 may permit a physician or other authorized user to program the operation of the LPs. Further, the external device 109 may be capable of causing the LPs 102 to perform functions necessary to complete certain methods of the present technology.

Now, considering the components of the external device 109 by reference to FIG. 9, operations of the external device 109 can be controlled by a CPU 902, which may be a generally programmable microprocessor or microcontroller or may be a dedicated processing device such as an Application Specific Integrated Circuit (ASIC) or the like. Software instructions to be performed by the CPU can be accessed via an internal bus 904 from a Read Only Memory (ROM) 906 and Random Access Memory (RAM) 930. Additional software may be accessed from a hard drive 908, floppy drive 910, and CD ROM drive 912, or other suitable permanent mass storage device. Depending upon the specific implementation, a Basic Input Output System (BIOS) is retrieved from the ROM by CPU at power up. Based upon instructions provided in the BIOS, the CPU “boots up” the overall system in accordance with well-established computer processing techniques.

Once operating, the CPU displays a menu of programming options to the user via an LCD display 914 or another suitable computer display device. To this end, the CPU may, for example, display a menu of specific programming parameters of the LP(s) 102 to be programmed or may display a menu of types of diagnostic data to be retrieved and displayed. In response thereto, the physician enters various commands via either a touch screen 916 overlaid on LCD display 914 or through a standard keyboard 918 supplemented by additional custom keys 920, such as an emergency WVI (EVVI) key. The EVVI key sets the LP(s) 102 to a safe WVI mode with high pacing outputs. This ensures life-sustaining pacing operation in nearly all situations but by no means is it desirable to leave cardiac stimulation device 100 in the EVVI mode at all times. The various types of displays, keys, and other input and output devices are examples of a user interface that can be used to implement certain embodiments described herein.

Typically, the physician initially controls the external device 109 to retrieve data stored within one or more of the LPs 102. To this end, CPU 902 transmits appropriate signals to a telemetry circuit 922, which provides components for directly interfacing with the LP(s) 102. The telemetry subsystem 922 can include its own separate CPU 924 for coordinating the operations of the telemetry subsystem 922. The main CPU 902 of the external device 109 communicates with telemetry subsystem CPU 924 via internal bus 904. The telemetry subsystem 922 additionally includes a telemetry circuit 926 for communicating with the LP(s). The telemetry subsystem 922 may utilize one or more types of communication technology to communicate with the LP(s), such as, but not limited to, conductive communication, RF communication, or inductive communication. Patient and device diagnostic data stored within the LP(s) 102 can be transferred to the external device 109. Further, the LP(s) 102 can be instructed to perform an electrode algorithms of the present invention, details of which are provided above. The CPU 902 can include a pacing optimization module 950 that is used to determine a preferred pacing parameter set, in accordance with certain embodiments of the present technology.

The external device 109 can also include a Network Interface Card (“NIC”) 960 to permit transmission of data to and from other computer systems via a router 962 and Wide Area Network (“WAN”) 964. Alternatively, the external device 109 might include a modem for communication via the Public Switched Telephone Network (PSTN). Depending upon the implementation, the modem may be connected directly to internal bus 904 and may be connected to the internal bus via either a parallel port 940 or a serial port 942. Data transmitted from other computer systems may include, for example, data regarding medication prescribed, administered, or sold to the patient.

The external device 109 receives data from the LP(s) 102, including parameters representative of the current programming state of the LP(s) 102. The external device 109 can also receive EGMs, samples thereof, and/or date indicative thereof from the LP(s) 102. Under the control of the physician, external device 109 displays the current programming parameters and permits the physician to reprogram the parameters. To this end, the physician enters appropriate commands via any of the aforementioned input devices and, under control of the CPU 902, the programming commands are converted to specific programming parameters for transmission to the LP(S) 102 to thereby reprogram the LP(s) 102. Prior to reprogramming specific parameters, the physician may control the external programmer to display any or all of the data retrieved from the LP(s) 102, including displays of ECGs, displays of electrodes that are candidates as cathodes and/or anodes, and statistical patient information. Any or all of the information displayed by external device 109 may also be printed using a printer 936.

A speaker 944 is included for providing audible tones to the user, such as a warning beep in the event improper input is provided by the physician. Telemetry subsystem 922 may additionally include an input/output circuit 946 which can control the transmission of analog output signals, such as ECG signals output to an ECG machine or chart recorder. Other peripheral devices may be connected to the external device 109 via parallel port 940 or a serial port 942 as well. Although one of each is shown, a plurality of Input Output (IO) ports might be provided.

With the external device 109 configured as shown, a physician or other authorized user can retrieve, process, and display a wide range of information received from the LP(s) 102 and reprogram the LP(s) 102, if needed. The descriptions provided herein with respect to FIG. 9 are intended merely to provide an overview of the operation of the example external device 109 and are not intended to describe in detail every feature of the hardware and software of the device and are not intended to provide an exhaustive list of the functions performed by the device.

Embodiments of the present technology have been described above with the aid of functional building blocks illustrating the performance of specified functions and relationships thereof. The boundaries of these functional building blocks have often been defined herein for the convenience of the description. Alternate boundaries can be defined so long as the specified functions and relationships thereof are appropriately performed. Any such alternate boundaries are thus within the scope and spirit of the claimed invention. For example, it would be possible to combine or separate some of the steps shown in FIGS. 6A and 6B. It would also be possible to reorder some of the steps shown in FIGS. 6A and 6B. For another example, it is possible to change the boundaries of some of the blocks shown in FIGS. 2, 8, and 9.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, it is noted that the term “based on” as used herein, unless stated otherwise, should be interpreted as meaning based at least in part on, meaning there can be one or more additional factors upon which a decision or the like is made. For example, if a decision is based on the results of a comparison, that decision can also be based on one or more other factors in addition to being based on results of the comparison.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the embodiments of the present technology without departing from its scope. While the dimensions, types of materials and coatings described herein are intended to define the parameters of the embodiments of the present technology, they are by no means limiting and are exemplary embodiments. Many other embodiments will be apparent to those of skill in the art upon reviewing the above description. The scope of the embodiments of the present technology should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112 (f), unless and until such claim limitations expressly use the phrase “means for” followed by a statement of function void of further structure.

What is claimed is:

1. A method for use with an implantable medical device (IMD) configured to deliver pacing pulses to cardiac tissue of a patient's heart, the method comprising:
determining a pacing impedance of the cardiac tissue that is to be paced by the IMD;
determining a first capture threshold of the cardiac tissue that is to be paced by the IMD when the cardiac tissue is paced using one or more pacing pulses having a first pulse width;
determining an estimate of a maximum membrane response for the cardiac tissue of the patient's heart, based on the pacing impedance, the first pulse width, and the first capture threshold with or without a specified safety margin applied;
wherein the first pulse width and the capture threshold with the specified safety margin applied collectively comprise a first pacing parameter set;
determining an iso-safety factor strength duration curve by determining, based on the first pacing parameter set and the estimate of the maximum membrane response, a plurality of further pacing parameter sets that each includes a respective different pulse width and a respective pacing amplitude having the specified safety margin applied;
determining a current or charge drain curve by determining, based on the pacing impedance, a respective current or charge drain value for each of the first pacing parameter set and the plurality of further pacing parameter sets; and
determining, based on the iso-safety factor strength duration curve and the current or charge drain curve, a preferred pacing parameter set that includes a preferred pulse width and a preferred pacing amplitude, which provides the specified safety margin at a lower current or charge drain than the first pacing parameter set.

2. The method of claim 1, wherein the determining the iso-safety factor strength duration curve includes:
determining a strength duration curve by determining, based on the first pacing parameter set and the estimate of the maximum membrane response, a plurality of further pacing parameter sets that each includes the respective different pulse width and a respective pacing amplitude without having the specified safety margin applied; and
applying the safety margin to respective pacing amplitudes of the first pacing parameter set and the plurality of further pacing parameter sets to thereby determine further respective pacing amplitudes with the safety margin applied, which are included in the iso-safety factor strength duration curve.

3. The method of claim 1, wherein the determining the iso-safety factor strength duration curve includes determining, based on the first pacing parameter set and the estimate of the maximum membrane response, the plurality of further pacing parameter sets that each includes the respective different pulse width and a respective pacing amplitude having the specified safety margin applied.

4. The method of claim 1, wherein the determining the estimate of the maximum membrane response for the cardiac tissue of the patient's heart is also based on a membrane time constant.

5. The method of claim 4, wherein the determining the iso-safety factor strength duration curve is performed without determining any additional capture threshold besides the first capture threshold.

6. The method of claim 5, wherein the membrane time constant is determined based on the first capture threshold and empirical patient population data.

7. The method of claim 4, further comprising:
determining one or more additional capture thresholds that correspond respectively to one or more additional pulse widths; and
determining the membrane time constant based on the first capture threshold and the one or more additional capture thresholds.

8. The method of claim 7, wherein the determining the membrane time constant based on the first capture threshold and the one or more additional capture thresholds includes performing error minimization using a nonlinear least squares regression algorithm.

9. The method of claim 4, further comprising:
determining one or more additional capture thresholds that correspond respectively to one or more additional pulse widths, thereby resulting in multiple capture thresholds with the specified safety margin applied being determined; and
wherein the determining the iso-safety factor strength duration curve is based on the multiple capture thresholds with the specified safety margin applied.

10. The method of claim 9, further comprising:
determining the membrane time constant based on the multiple capture thresholds with the specified safety margin applied; and
using the membrane time constant to produce the iso-safety factor strength duration curve.

11. The method of claim 10, wherein the determining the membrane time constant based on the multiple capture thresholds with the specified safety margin applied includes performing error minimization using a nonlinear least squares regression algorithm.

12. The method of claim 1, further comprising:
the IMD delivering pacing pulses, having the preferred pulse width and the preferred pacing amplitude, to the cardiac tissue of the patient's heart.

13. The method of claim 12, further comprising:
displaying, via a user interface of an external device that is configured to communicate with the IMD, information indicative of the preferred pacing parameter set that includes the preferred pulse width and the preferred pacing amplitude is displayed to a user; and
programming the IMD to use the preferred pacing parameter set.

14. The method of claim 1, wherein the determining the preferred pacing parameter set that includes the preferred pulse width and the preferred pacing amplitude is performed by an external device that is configured to communicate with the IMD.

15. The method of claim 1, wherein the determining the preferred pacing parameter set that includes the preferred pulse width and the preferred pacing amplitude is performed by the IMD.

16. The method of claim 1, wherein the IMD includes a battery that outputs a battery voltage and a charge pump that can apply one of a plurality of multiplier factors to the battery voltage to achieve a range of pacing amplitudes, and wherein:
the determining the current or charge drain curve is performed in a manner that accounts for the plurality of multiplier factors and causes the current or charge drain curve to have multiple inflection points.

17. The method of claim 1, wherein the IMD comprises a leadless pacemaker (LP) that includes at least two electrodes that are used to deliver the pacing pulses to the cardiac tissue of a patient's heart.

18. The method of claim 17, wherein the method is performed for each of a plurality of LPs to thereby determine a different said preferred pacing parameter set for each of the plurality of LPs.

19. The method of claim 1, wherein the IMD comprises a pacemaker to which one or more leads having one or more electrodes is/are attached, and wherein the method is performed for each of a plurality of different electrode combinations to thereby determine a different said preferred pacing parameter set for each of the plurality of different electrode combinations.

20. A system including or for use with an implantable medical device (IMD) configured to deliver pacing pulses to cardiac tissue of a patient's heart, the system comprising one or more processors configured to:
determine a pacing impedance of the cardiac tissue that is to be paced by the IMD;
determine a first capture threshold of the cardiac tissue that is to be paced by the IMD when the cardiac tissue is paced using one or more pacing pulses having a first pulse width;
determine an estimate of a maximum membrane response for the cardiac tissue of the patient's heart, based on the pacing impedance, the first pulse width, and the first capture threshold with or without a specified safety margin applied;
wherein the first pulse width and the capture threshold with the specified safety margin applied collectively comprise a first pacing parameter set;
determine an iso-safety factor strength duration curve by determining, based on the first pacing parameter set and the estimate of the maximum membrane response, a plurality of further pacing parameter sets that each includes a respective different pulse width and a respective pacing amplitude having the specified safety margin applied;
determine a current or charge drain curve by determining, based on the pacing impedance, a respective current or charge drain value for each of the first pacing parameter set and the plurality of further pacing parameter sets; and
determine, based on the iso-safety factor strength duration curve and the current or charge drain curve, a preferred pacing parameter set that includes a preferred pulse width and a preferred pacing amplitude, which provides the specified safety margin at a lower current or charge drain than the first pacing parameter set.

21. The system of claim 20, wherein the one or more processors is/are configured to:
determine a strength duration curve by determining, based on the first pacing parameter set and the estimate of the maximum membrane response, a plurality of further pacing parameter sets that each includes the respective different pulse width and a respective pacing amplitude without having the specified safety margin applied; and
apply the safety margin to respective pacing amplitudes of the first pacing parameter set and the plurality of further pacing parameter sets to thereby determine further respective pacing amplitudes with the safety margin applied, which are included in the iso-safety factor strength duration curve.

22. The system of claim 20, wherein the one or more processors is/are configured to determine, based on the first pacing parameter set and the estimate of the maximum membrane response, the plurality of further pacing parameter sets that each includes the respective different pulse width and a respective pacing amplitude having the specified safety margin applied, to thereby determine the iso-safety factor strength duration curve.

23. The system of claim 20, wherein the one or more processors is/are configured to determine the estimate of the maximum membrane response for the cardiac tissue of the patient's heart is also based on a membrane time constant.

24. The system of claim 20, wherein the system is configured to program the IMD to deliver pacing pulses, having the preferred pulse width and the preferred pacing amplitude, to the cardiac tissue of the patient's heart.

25. The system of claim 20, the system further comprising:
a plurality of implantable electrodes; and
an implantable pulse generator electrically couplable to the implantable electrodes;
wherein at least one of the one or more processors are configured to control the pulse generator to deliver pacing pulses to cardiac tissue via at least two of the implantable electrodes.

26. The system of claim 25, wherein the system comprises the IMD that includes or is coupled to the plurality of implantable electrodes.

27. The system of claim 26, wherein the one or more processors is/are included in the IMD.

28. The system of claim 20, wherein the system comprises an external device and at least one of the one or more processors configured to determine the preferred pacing parameter set is/are included in the external device.

29. The system of claim 28, wherein the external device is configured to communicate with the IMD and to program the IMD to use the preferred pacing parameter set to pace the cardiac tissue.

30. The system of claim 29, wherein:
information indicative of the preferred pacing parameter set that includes the preferred pulse width and the preferred pacing amplitude is displayed to a user via a user interface of the external device that is configured to communicate with the IMD; and
the external device is configured to program the IMD to use the preferred pacing parameter set in response to the user accepting the preferred pacing parameter set via the user interface.
